(19) United States
(12) Patent Application Publication
(10) Pub. No.: US 2025/0342916 A1
(43) Pub. Date: Nov. 6, 2025

(54) Methods and Systems for Predicting Crystal Structures

(71) Applicant: Good Chemistry Inc., Tarrytown, NY (US)

(72) Inventors: Kevin RYCZKO, Gatineau (CA); Amit KADAN, Vancouver (CA); Takeshi YAMAZAKI, Vancouver (CA)

(21) Appl. No.: 18/940,746

(22) Filed: Nov. 7, 2024

Related U.S. Application Data
(60) Provisional application No. 63/599,039, filed on Nov. 15, 2023.

(51) Int. Cl. G16C 20/30; G16C 20/50; G16C 20/70

(52) U.S. Cl. CPC G16C 20/30; G16C 20/70; G16C 20/50

(57) ABSTRACT

Methods and systems for predicting crystal structures. One of the methods includes providing an indication of the one or more molecules; generating a set of crystal structures based on the indication; generating a reliability metric of a machine learning model for generating a property metric for each crystal structure in the set; calculating the property metric for each crystal structure in the set of crystal structures to generate a set of property metrics, by (i) using the machine learning model if the reliability metric for the crystal structure is within a predetermined threshold, and (ii) using a ground truth calculation of the crystal structure if the reliability metric for the crystal structure is not within the predetermined threshold; and taking an action based on the set of crystal structure indications for the one or more molecules.

### CROSS-REFERENCE TO RELATED APPLICATIONS

This application claims the benefit under 35 U.S.C. § 119(e) of the filing date of U.S. Patent Application No. 63/599,039, entitled “Methods and Systems for Predicting Crystal Structures, which was filed on Nov. 15, 2023, and which is incorporated here by reference in its entirety.

### BACKGROUND

### Technical Field

This specification relates to computer-based organic crystal structure prediction.

### Background

The discovery of low-energy molecular crystals plays an important role in developing new drugs and electronic devices. In some cases, the discovery process can take IO years or more and more than 2 billion US dollars. Computer-aided molecular crystal structure prediction (CSP) is a faster and less expensive approach compared to a laboratory-based process.

### SUMMARY

This specification describes technologies for predicting organic crystal structures using machine learning. These technologies generally involve using a machine learning (ML) model to score crystal candidates. As the ML model can be configured to quantify its uncertainty in making a prediction, an active learning method can be implemented to improve the accuracy of the ML model. The CSP tool can become more accurate with every active learning iteration as informative data points are identified and sampled using rigorous methods, which can be used to train or fine-tune the ML model.

In an exemplary implementation of the method (or a system implemented thereof), the method can involve receiving an input including an indication of a molecule and generating a sorted list of crystal structures in response to receiving the input. In particular, the input can include an indication that identifies a molecule for which the crystal structure is desired to be predicted. The technologies described herein involve generating a set of crystal structures based on the input, using a machine learning model to calculate a property metric for each crystal structure in the set, and determining how reliable each calculated property metric is based on a reliability metric calculated for each property metric. Property metrics calculated by the machine learning model that are determined to not be reliable are then calculated a ground truth calculation instead. Meanwhile, those property metrics that are calculated using a ground truth calculation are then used to train the machine learning model in order to improve its capacity to calculate property metrics for future sets of crystal structures. Finally, the set of calculated property metrics is used to generate a sorted list of crystal structures corresponding to the given molecule, where the list can be sorted based on the set of calculated property metrics.

In general, one innovative aspect of the subject matter described in this specification can be embodied in methods that include the actions of providing an indication of the one or more molecules; generating a set of crystal structures based on the indication; generating a reliability metric of a machine learning model for generating a property metric for each crystal structure in the set of crystal structures; calculating the property metric for each crystal structure in the set of crystal structures to generate a set of property metrics, by (i) using the machine learning model if the reliability metric for the crystal structure is within a predetermined threshold, and (ii) using a ground truth calculation of the crystal structure if the reliability metric for the crystal structure is not within the predetermined threshold; and taking an action based on the set of crystal structure indications for the one or more molecules, wherein the set of crystal structure indications is based on the set of crystal structures and the set of property metrics.

Other embodiments of this aspect include corresponding computer systems, apparatus, and computer programs recorded on one or more computer storage devices, each configured to perform the actions of the methods. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

The foregoing and other embodiments can each optionally include one or more of the following features, alone or in combination. In particular, one embodiment includes all the following features in combination.

In some implementations, the property comprises density, solubility, stability, ADMET, or any combination thereof. In some implementations, the property metric is an energy metric. In some implementations, the energy metric is potential energy. In some implementations, the energy metric is free energy.

In some implementations, the machine learning model comprises a neural network.

In some implementations, the reliability metric is indicative of an accuracy of the machine learning algorithm for generating the property metric.

In some implementations, calculating the property metric for each crystal structure in the set of crystal structures to generate a set of property metrics further comprises, if the reliability metric for the crystal structure is not within the predetermined threshold, storing the crystal structure and the calculated property metric in a training dataset. In some implementations, the method further comprises preparing the training dataset for training the machine learning model. In some implementations, the method further comprises training the machine learning model using the training dataset. In some implementations, the training dataset comprises at least 10, 100, 1 k, 10 k, 100 k, or 1M crystal structures and calculated energy metrics.

In some implementations, the set of crystal structure indications comprises a set of polymorphic crystal structure indications, and the method further comprises sorting the set of polymorphic crystal structure indications based on the set of property metrics to output a report comprising a sorted set of polymorphic crystal structures.

In some implementations, the ground truth calculation is based on interatomic interactions. In some implementations, the interatomic interactions comprise potential energy functions. In some implementations, the potential energy functions comprise one or more functions from OPLS, AMBER, CHARM, UFF, neural-network potential energy functions, or any combination thereof.

In some implementations, the ground truth calculation is based on electronic interactions. In some implementations, the ground truth calculation is computed using any one of a molecular dynamics method, a Monte Carlo method, or a quantum mechanical method.

In some implementations, taking an action comprises forwarding data characterizing the set of crystal structure indications for display.

In some implementations, providing an indication of the one or more molecules comprises receiving an indication from a generative machine learning model.

Another innovative aspect of the subject matter described in this specification can be embodied in an active learning method for reporting a set of crystal structure indications for one or more molecules, comprising: providing an indication of the one or more molecules; generating a set of crystal structures based on the indication; generating a reliability metric for generating a property metric for each crystal structure in the set of crystal structures; calculating the property metric for each crystal structure in the set of crystal structures to generate a set of energy metrics, by using a ground truth calculation of the crystal structure if the reliability metric for the crystal structure is not within the predetermined threshold; storing the indication, the set of crystal structures, the reliability metric, the property metric, or any combination thereof in a training dataset; and training a machine learning algorithm using the training dataset, wherein the machine learning algorithm is used to perform one or more of: providing an indication of the one or more molecules; generating a set of crystal structures based on the indication; generating a reliability metric for generating a property metric for each crystal structure in the set of crystal structures; and calculating the property metric for each crystal structure in the set of crystal structures to generate a set of energy metrics, by using a ground truth calculation of the crystal structure if the reliability metric for the crystal structure is not within the predetermined threshold.

The subject matter described in this specification can be implemented in particular embodiments so as to realize one or more of the following advantages.

The technologies described in the present disclosure allow for the generation of crystal structures for a given molecule that are relatively likely to match an experimentally-derived structure for the molecule. This specification also discloses a machine learning model that describes the properties of the generated crystal structures. In particular, the methods disclosed herein include adding property metrics and corresponding crystal structures to a training dataset that will later be used to train (or retrain) a machine learning model, if the initial calculation of the property metrics by the machine learning model was determined to be unreliable. This enables the machine learning model to be trained on those crystal structures and property metrics with respect to which it was initially unreliable, e.g., unreliable beyond a specified threshold. Thus, areas of low reliability of the machine learning model can be identified and improved more efficiently.

Additionally, the present disclosure provides systems and methods for generating latent representations of crystal structures that can enable more efficient discovery, design, and/or development of useful crystal structures. The systems and methods disclosed herein can provide improved data efficiency such that less data (or, more fundamentally, less information) can be used to generate useful crystal structures.

The techniques described herein are highly advantageous when compared with conventional techniques involving random generation to predict crystal structures. For example, a number of blind tests have been conducted in which the performance of various techniques for generating crystal structure candidates for a number of different target molecules were compared. The techniques employed in the blind tests also ranked the generated crystal structure candidates according to a predicted likelihood of the generated crystal structure candidate matching the experimentally-derived crystal structure. The ranking performance of the techniques was also compared.

In the blind tests, the techniques described herein generated two more crystal structure candidates that matched an experimentally-derived crystal structure (representing a 20% increase), as compared with an example random generation technique. Additionally, the techniques described herein ranked generated crystal structure candidates within the top 100 of the ranked crystal structure candidates for two additional target molecules (representing a 100% increase) in comparison with the example random generation technique. Finally, the techniques described herein ranked generated crystal structure candidates within the top 500 of the ranked crystal structure candidates for five additional target molecules (representing a 250% increase) in comparison with the example random generation technique.

Additionally, in some implementations, the techniques described herein allow for making incremental improvements to current structures rather than starting from scratch (i.e. random generation). In such implementations, the techniques described herein can surpass the success rate of random generation alone. In particular, the techniques described herein require 10 to 100 times fewer structures in the initial pool relative to the example random generation technique, exhibiting that the techniques described herein can effectively explore the potential energy landscape.

The techniques described herein are also much faster than the example random generation technique. For example, to generate a match for a particular target molecule (xxiii-A) with the example random generation technique can take >10,000 CPU hours, whereas with the techniques described herein, a match can be generated in under 3000 CPU hours.

The techniques for ranking crystal structures that are described herein also provide an advantage over other methods for ranking crystal structures. For example, the techniques described herein approach speeds similar to methods employing force-fields for ranking, but are orders of magnitude faster than methods using ab initio methods. Additionally, the aspects of the techniques described herein that incorporate machine learning for ranking in CSP allow for speeds almost as fast as force-field-based methods, while also approaching the accuracy achieved by quantum chemistry methods.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is an example crystal structure prediction (CSP) system.

FIG. 2 is a flow diagram of an example process for reporting a set of crystal structure indications for one or more molecules.

FIG. 3 is a diagram illustrating views of example inputs and outputs of steps in the process disclosed herein.

FIGS. 4A-4E are exemplary web-based graphical user interfaces with which a user can interact in order to cause a system to carry out the techniques described herein.

FIG. 5 is a set of output crystal structure indications generated using the techniques described herein.

FIG. 6 is a graph illustrating the performance of the techniques described herein on a number of blind tests.

FIG. 7 is a graph illustrating the time taken to generate predicted structures of various target molecules using the techniques described herein.

FIG. 8 is a block diagram depicting an exemplary machine that includes a computer system (e.g., a processing or computing system) within which a set of instructions can execute for causing a device to perform the techniques described herein.

Like reference numbers and designations in the various drawings indicate like elements.

### DETAILED DESCRIPTION

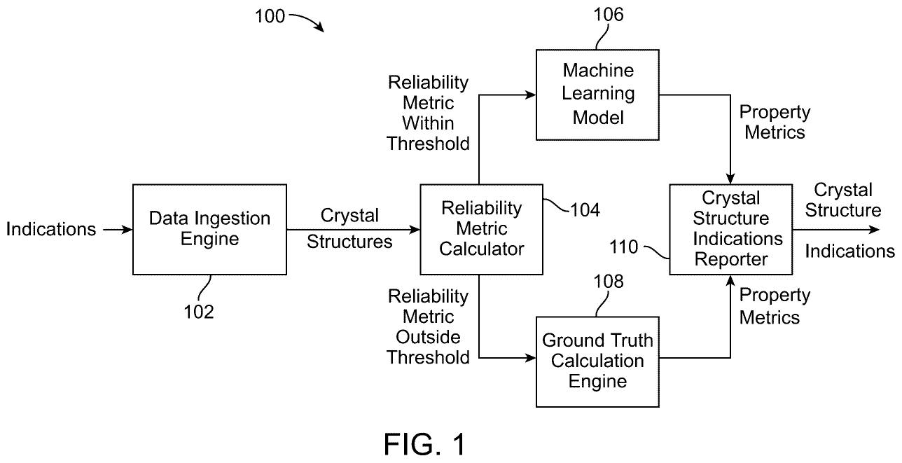

FIG. 1 is an example crystal structure prediction (CSP) system 100. The CSP system 100 includes a data ingestion engine 102, a reliability metric calculator 104, a machine learning (ML) model 106, a ground truth calculation engine 108, and a crystal structure indication reporter 110.

The data ingestion engine 102 receives one or more indications of one or molecules and generates a set of crystal structures for the one or more molecules based on the one or more indications. In some implementations, the one or more molecules can be a pharmaceutical or, more specifically, a pharmaceutical salt. An indication can be information that represents one or more molecules. The indication can be the property of a crystal structure of the one or more molecules. The indication can be used to identify the one or more molecules. The indication can include an identifier, such as a textual identifier, which can provide specifics of the molecular structure of the one or more molecules at various levels of detail.

For example, the identifier can be a chemical formula indicating the constituent atoms of a molecule. The identifier can be a structural formula, which can be encoded in a programming object or a datafile. The identifier can indicate the structural formula, e.g., SMILES or SELFIES. The identifier can indicate the structural formula as well as the specific stereoisomer or the specific rotamer, e.g., InChi. The specific form or format in which the identifier can be provided in the indication can vary (e.g., the identifier can be provided as a string, a graph or an adjacency matrix, a data file such as a PDB, binary file, provided in memory, etc.). As an example, the identifier can include one or more 3D structures of the one or more molecules. As an additional example, the identifier can include one or more data objects of the one or more molecules.

Geometric arrangement of atoms, electrons, and/or motifs in a molecule or a crystal structure can be described in different manners and different levels of precision in the indication. Various formats and precisions can impart unique value in terms of training and utilizing a CSP model. Formats such as MOL2, PDB, MOL, PDBQ/PDBQT, SDF, SMILES, SELFIES, InChl, Chemdraw (CDX, CDXML), CIF, CML, XML, ASNI, PARM, CRD, or TRJ format can be used. The indication can provide coordinates, e.g., Cartesian coordinates, or internal coordinates (e.g., bonds, angles, and dihedrals). The indication can be in human readable form, or can be encoded in binary or other suitable data types. The indication can include one or a plurality of molecules. In some implementations, a “representation”, a “descriptor”, an “identifier”, can be used synonymously. In some implementations, an indication can be used as feature values to a neural network.

In some implementations, the indication can be provided by one or more users. For example, one or more users can be interested in one or more crystal structures of a specific compound, or a composition comprising multiple compounds. The one or more users can provide the indication to the CSP system 100, which can return results relevant for the one or more users.

In some implementations, the present disclosure provides a web-based graphical user interface comprising a first field configured to receive a user query. The user query can comprise the indication of the one or more molecules. The web-based graphical user interface can comprise a second field configured to return a set of crystal structure indications for the one or more molecules.

In some implementations, the indication can be provided by a generative machine learning model. The generative machine learning model can include a genetic algorithm. For example, an active learning method can be configured to provide one or more indications which, if added to a training dataset or a fine-tuning dataset, provides efficient sampling of the problem space so that CSP capabilities can be enhanced over iterations. The active learning method can be configured to provide selectively the indication selected from a molecular space where the reliability metric is expected to be low, and therefore, the information gain from obtaining a data point at that region of the molecular space is high. Over time, the active learning method can provide any number of indications from various regions of the molecular space, e.g., at least 10, 100, 1 k, 10 k, 100 k, or 1M indications. The amount of computational power required to obtain data points for a large number of indications can be provided by a scalable computing infrastructure, e.g., cloud computing. The generative machine learning model can alternatively or additionally include a neural network.

In some implementations, a machine learning model may provide an indication for one or more specific molecules of interest in order to satisfy a certain objective. For example, a machine learning model can be assigned a task of finding certain molecular structures or compositions which would result in a crystal structure. Thus, the machine learning algorithm may provide an initial set of one or more indications in a chemical space to be explored by the CSP model, and return results of the exploration. When the results of the exploration do not yet satisfy the objective, the algorithm may provide another set of one or more indications which may have a higher probability of satisfying the objective. Therefore, the machine learning model can guide the CSP model towards finding a solution to a specific objective. The specific objective can be a desirable property. The specific objective can be, for example, to find a composition of a pharmaceutically interesting compound or salt thereof with one or more excipients that would crystallize. The specific objective can be, finding a polymorph of a crystal of a pharmaceutically interesting compound or a salt thereof with one or more excipients. The machine learning model can be a neural network. In some implementations, a genetic algorithm may provide an indication for one or more specific molecules of interest in order to satisfy a certain objective.

In some implementations, a property can be used as an objective of exploration by the CSP algorithm. The property can be density, solubility, stability, ADMET (absorption, distribution, metabolism, and excretion), or any combination thereof. A property can be used as a metric of accuracy of the CSP algorithm. For example, the property can be an energy metric. The energy metric can be potential energy or free energy.

In some implementations, the indication can be provided by random sampling.

In some implementations, an indication can include one or more electron configurations. In some implementations, an electron configuration can include one or more atomic orbitals, one or more molecular orbitals, or both. In some implementations, an electron configuration can include valence electrons of an atom. In some implementations, an electron configuration can include a character of an electron (e.g., s, p, d, f, and any mixtures thereof). In some implementations, an electron configuration can include an electron spin. In some implementations, an electron configuration can include electron density. In some implementations, an electron configuration can be represented in various basis functions, including but not limited to, atomic orbitals, molecular orbitals, or plane waves.

Within various cheminformatic formats can be differently encoded information. In some implementations, an indication can include one or more atomistic representations. In some implementations, an atomistic representation can include the relative cartesian coordinates of atoms to each other. In some implementations, an atomistic representation can include the relative cartesian coordinates of atoms to an arbitrary point. In some implementations, an atomistic representation can include thermodynamic estimations of values such as salvation energy, potential energy of bond lengths, bond angles, dihedral angles, 1-4 intramolecular interaction energies, intramolecular energies among adjacent bond angles, hydrogen bonding energies, and non-bonded interaction energies. In some implementations, an atomistic representation can include atom type definitions and generalizations. In some implementations, an atomistic representation can include polarizability parameters. In some implementations, an atomistic representation can include Lennard-Jones van der Waal parameters. In some implementations, an atomistic representation can include electrostatic charge parameters. In some implementations, an atomistic representation can include bond length, bond angle, and dihedral force constants. In some implementations, an atomistic representation can include bond length, bond angle, and dihedral equilibrium values. In some implementations, an atomistic representation can include dihedral phase and periodicity force constants.

In some implementations, indications of molecular structures or crystal structures can be screened based on a predicted property metric. The predicted property metric of an indication may be used to include or exclude the indication in a screening method. For example, a measure of solubility (e.g., free energy of salvation) in water or a biologically relevant solution (e.g., plasma, stomach acid) of a chemical system described by the indication can be predicted by a method or system of the present disclosure, which can be used as a basis for including or excluding an indication from a candidate set for experimentation. Likewise, the predicted property metric can be any metric relevant for the particular task, which can be set by the user. In pharmaceutical discovery applications, ADMET (Absorption, Metabolism, Distribution, Excretion, or Toxicological) properties may be relevant property metrics.

Upon receiving the one or more indications of one or molecules, the data ingestion engine 102 generates a set of crystal structures based on the one or more indications. The data ingestion engine 102 can generate a crystal structure in the set of crystal structures based on the one or more indications by generating a set of conformers of the one or more molecules.

In some implementations, the data ingestion engine 102 can generate the set of conformers for each crystal structure in the set of crystal structures at the same time. In some implementations, the data ingestion engine 102 can generate the set of conformers for each crystal structure in the set of crystal structures one at a time.

In some implementations, the data ingestion engine 102 can generate a crystal structure in the set of crystal structures based on the one or more indications by arranging one or more conformers of the set of conformers in space. The space can be 3D space, which can have a Cartesian coordinate system or be convertible into a Cartesian coordinate system. The space can include a Bravais lattice, unit cell parameters, or a space group. The arrangement of the one or more conformers in space can include replicas of one conformer among the one or more conformers. Additionally or alternatively, the arrangement can include a plurality of different conformers. The plurality of different conformers can be of the same molecule in the one or more molecules. The plurality of different conformers can be of different molecules in the one or more molecules.

In some implementations, the data ingestion engine 102 can generate one or more conformers in the set of conformers using cheminformatics tools or computational chemistry calculations or simulations. For example, the data ingestion engine 102 can generate one or more conformers in the set of conformers using cheminformatics tools, such as RDKit™ or OpenBabel, to generate various conformers of a molecule given an identifier of the molecule (e.g., given a SMILES string of a molecule, various conformers of the molecule can be generated, the conformers varying in bond lengths, angles, torsional angles, etc.).

In some implementations, the data ingestion engine 102 can use computational chemistry calculations or simulations to generate one or more conformers in the set of conformers. The computational chemistry calculations or simulations can employ one or more of a force-field-based method or an ab initio method. For example, the data ingestion engine 102 can use Monte Carlo or molecular dynamics methods to generate an ensemble of conformers at various certain temperatures, pressures, and chemical potentials.

In some implementations, the data ingestion engine 102 can use computational chemistry calculations or simulations to optimize the geometry of one or more conformers in the set of conformers. The data ingestion engine 102 can perform such geometry optimization of the one or more conformers if the one or more conformers were generated using cheminformatics tools, or if the one or more conformers were generated using computational chemistry calculations or simulations.

In some implementations, the data ingestion engine 102 can optimize an arrangement of conformers in one or more crystal structures of the set of crystal structures. The data ingestion engine 102 can optimize an arrangement of conformers in one or more crystal structures of the set of crystal structures based on an empirical force-field based method, an ab initio method (any suitable method among methods of varying degrees of detail, from coupled cluster to DFT), a machine learning method, or a combination thereof. The optimization of the arrangement of conformers in one or more crystal structures can involve displacing atoms in the arrangement to reduce an energy metric for each of the one or more crystal structures. The data ingestion engine 102 can use an energy metric as a metric for the stability of a crystal structure, as finding a local or global minimum of the energy metric could be indicative of the stability of the crystal structure. The energy metric can be, e.g., based on potential energy or free energy.

In implementations in which the energy metric is based on potential energy, the data ingestion engine 102 can calculate the potential energy directly from the positions of atoms using one or more of an empirical, machine learning, or an ab initio method. In implementations in which the energy metric is based on free energy, the data ingestion engine 102 can calculate the free energy by taking into account entropic effects, e.g., contributions from vibrational modes (Hessian), via thermodynamic integration methods using molecular dynamics, etc.

In some implementations, the one or more molecules include building blocks that form chemical bonds between them and generate materials, such as metal-organic framework and covalent organic frameworks. In such implementations, the crystal structure generated by the data ingestion engine 102 can include one or more bonds between the set of conformers of the one or more molecules. The bonds can be covalent bonds. In some cases, the one or more molecules are building blocks of covalent organic frameworks. In some implementations, the one or more molecules can include one or more metallic atoms. In such implementations, the crystal structure can include one or more bonds between the set of conformers of the one or more molecules and the one or more metal atoms. In some implementations, the one or more molecules are building blocks of metal organic frameworks.

In some implementations, the data ingestion engine 102 can generate each crystal structure in the set of crystal structures at the same time. In some implementations, the data ingestion engine 102 can generate each crystal structure in the set of crystal structures one at a time. In some implementations, the data ingestion engine 102 can generate each crystal structure in the set of crystal structures in parallel, optionally using one or more distributed processes. The one or more distributed processes can be coordinated through a message passing interface (MPI).

The reliability metric calculator 104 receives the set of crystal structures for the one or more molecules from the data ingestion engine 102 and, based on the received set of crystal structures, generates a reliability metric of a machine learning algorithm for generating a property metric for each crystal structure in the set of crystal structures. The reliability metric for each crystal structure can be an indication of the accuracy of the machine learning model 106 in generating a property metric for the crystal structure, the process of which is described in further detail below. In particular, the reliability metric falling within a predetermined threshold can be indicative of a high accuracy of the machine learning model 106 in generating a property metric for the crystal structure. The reliability metric falling outside of the predetermined threshold can be indicative of a low accuracy of the machine learning model 106 in generating a property metric for the crystal structure.

In some implementations, the reliability metric calculator 104 generates the reliability metric for each crystal structure in the set of crystal structures at the same time. In some implementations, the reliability metric calculator 104 generates the reliability metric for each crystal structure in the set of crystal structures one at a time.

In some implementations, the reliability metric calculator 104 uses a second machine learning algorithm to generate a reliability metric for each crystal structure in the set of crystal structures. The second machine learning algorithm that calculates the reliability metric can be included in a machine learning algorithm used by the machine learning model 106 in generating a property metric for the crystal structure. In some implementations, the second machine learning algorithm can calculate the reliability metric simultaneously while the machine learning algorithm used by the machine learning model 106 generates a property metric for the crystal structure. In some implementations, the second machine learning algorithm can calculate the reliability metric after the machine learning algorithm used by the machine learning model 106 generates a property metric for the crystal structure.

The second machine learning algorithm can be configured to receive features of a crystal structure. The features can include 3D coordinates, atom types, and/or bonding information. For example, bonding information can include interatomic distances in a bond (ij), angles (ijk), dihedrals (ijkl), or any combination thereof. The features can include a Bravais lattice indication, a space group indication, or any other feature indicative of a shape, size, or symmetry of a crystal structure.

The second machine learning algorithm can include a plurality of machine learning algorithms. Each machine learning algorithm in the plurality of machine learning algorithms can be configured to receive features of a crystal structure as described above. Each machine learning algorithm in the plurality of machine learning algorithms can generate an output for each crystal structure. In some implementations, each machine learning algorithm in the plurality of machine learning algorithms calculates a property metric of interest for each crystal structure. The property metric of interest can be the same property metric that is generated by the machine learning model 106, the process of which is described in further detail below.

The reliability metric calculator 104 can generate the reliability metric for a given crystal structure by comparing the outputs of the plurality of machine learning algorithms for the crystal structure. For example, the reliability metric for a given crystal structure generated by the reliability metric calculator 104 can be based on how close the outputs of the plurality of machine learning algorithms for the crystal structure are in value. In particular, the reliability metric calculator 104 can generate a reliability metric for a given crystal structure indicating a high accuracy of the machine learning algorithm used by the machine learning model 106 if the outputs of the plurality of machine learning algorithms for the crystal structure have a standard deviation below a given threshold. The reliability metric calculator 104 can generate a reliability metric for a given crystal structure indicating a low accuracy of the machine learning algorithm used by the machine learning model 106 if the outputs of the plurality of machine learning algorithms for the crystal structure do not fall within a given range.

If the reliability metric for a given crystal structure calculated by the reliability metric calculator 104 is within a predetermined threshold, the machine learning (ML) model 106 uses the machine learning algorithm to calculate a property metric for the crystal structure based on one or more crystal structures in the set of crystal structures generated by the ingestion engine 102.

The machine learning model 106 can include one or more of various machine learning models. For example, the machine learning model 106 can include one machine learning model. As another example, the machine learning model 106 can include a plurality of machine learning models.

One or more of the various machine learning models of the machine learning model 106 can be configured to receive features of a crystal structure. The features can include 3D coordinates, atom types, bonding information. For example, bonding information can include interatomic distances in a bond (ij), angles (ijk), dihedrals (ijkl), or any combination thereof. The features can include a Bravais lattice indication, a space group indication, or any other feature indicative of a shape, size, or symmetry of a crystal structure.

In some implementations, the machine learning model 106 can include a neural network model. In some implementations, the machine learning model 106 can include a random forest model. In some implementations, the machine learning model 106 can include a manifold learning model. In some implementations, the machine learning model 106 can employ a hyperparameter optimization model to optimize the hyperparameters of the machine learning model 106. In some implementations, the machine learning model 106 can include an active learning model.

In some implementations, the machine learning model 106 can include a graph model. In some implementations, the graph model can include a directed graph or an undirected graph. A graph, graph model, and graphical model can refer to a method of conceptualizing or organizing information into a graphical representation comprising nodes and edges. In some implementations, a graph can refer to the principle of conceptualizing or organizing data, wherein the data can be stored in a various and alternative forms such as linked lists, dictionaries, spreadsheets, arrays, in permanent storage, and in transient storage, and is not limited to specific implementations disclosed herein.

In a preferred embodiment, the machine learning model 106 can be an ensemble of atomistic feed-forward neural networks operating over atomic environment vectors (AEVs). The machine learning model 106 can include a neural network comprising various architectures, loss functions, optimization algorithms, priors, and various other neural network design choices. In some implementations, the machine learning model 106 can include a neural network. In some implementations, the machine learning model 106 can include an autoencoder. In some implementations, the machine learning model 106 can include a generative model. In some implementations, the machine learning model 106 can include a variational autoencoder. In some implementations, the machine learning model 106 can include a generative adversarial network. In some implementations, the machine learning model 106 can include a flow model. In some implementations, the machine learning model 106 can include an autoregressive model. In some implementations, the machine learning model 106 can include a neural network. In some implementations, the machine learning model 106 can include a neural network with fully connected layers. In some implementations, the machine learning model 106 can include a neural network with convolutional layers. In some implementations, the machine learning model 106 can include a neural network with message-passing layers. In some implementations, the machine learning model 106 can include a neural network with a bottleneck layer. In some cases, a layer can include an attention mechanism, a generalized message-passing graph neural network, or both. In some cases, a generalized message-passing graph neural network includes a graph convolutional neural network.

In some implementations, the machine learning model 106 can include a neural network with residual blocks. In some implementations, the machine learning model 106 can include a neural network with attention. In some implementations, the machine learning model 106 can include a neural network with one or more non-linearities. In some implementations, the machine learning model 106 can include a neural network with one or more dropout layers. In some implementations, the machine learning model 106 can include a neural network with one or more batch normalization layers.

In some implementations, the machine learning model 106 can include a regression loss function. In some implementations, the machine learning model 106 can include a logistic loss function. In some implementations, the machine learning model 106 can include a variational loss. In some implementations, the machine learning model 106 can include a prior. In some implementations, the machine learning model 106 can include a Gaussian prior. In some implementations, the machine learning model 106 can include a non-Gaussian prior. In some implementations, the machine learning model 106 can include an adversarial loss. In some implementations, the machine learning model 106 can include an autoencoding loss.

In some implementations, the machine learning model 106 is trained with the Adam optimizer. In some implementations, the machine learning model 106 is trained with the stochastic gradient descent optimizer. In some implementations, the hyperparameters of the machine learning model 106 are optimized with Gaussian Processes. In some implementations, the machine learning model 106 is trained with train/validation/test data splits. In some implementations, the machine learning model 106 is trained with k-fold data splits, with any positive integer fork.

The machine learning model 106 can include a variety of manifold learning algorithms. In some implementations, the machine learning model 106 can include a manifold learning algorithm. In some implementations, the manifold learning algorithm is principle component analysis. In some implementations, the manifold learning algorithm is a uniform manifold approximation algorithm. In some implementations, the manifold learning algorithm is an isomap algorithm. In some implementations, the manifold learning algorithm is a locally linear embedding algorithm. In some implementations, the manifold learning algorithm is a modified locally linear embedding algorithm. In some implementations, the manifold learning algorithm is a Hessian eigen-mapping algorithm. In some implementations, the manifold learning algorithm is a spectral embedding algorithm. In some implementations, the manifold learning algorithm is a local tangent space alignment algorithm. In some implementations, the manifold learning algorithm is a multi-dimensional scaling algorithm. In some implementations, the manifold learning algorithm is a t-distributed stochastic neighbor embedding algorithm (t-SNE). In some implementations, the manifold learning algorithm is a Barnes-Hut t-SNE algorithm.

Various molecular modeling techniques can be used to train and/or develop the machine learning model 106. The effectiveness of the machine learning model 106 can be dependent on the quality, quantity, and diversity of the chemical systems that serve as inputs to the machine learning model 106 during training. Various experimental and computational methods can be used to generate useful inputs for machine learning, including quantum mechanical calculations, experimental characterizations, and molecular dynamics. In some cases, quantum mechanical calculations and/or approximations can yield accurate descriptors of key molecular properties such as charge distribution, optimal molecular conformations, and transition energies between conformations, macroscopic properties (e.g., salvation free energies). Quantum mechanical approximations can range from Hartree-Fock and DFT to highly expensive calculations such as coupled cluster. Molecular dynamics can be used as an input source for useful training data for machine learning. Techniques such as thermodynamic integration can provide free energies of salvation, stability of a crystal structure, etc.

In some implementations, the methods of the disclosure further include reducing one or more molecular or crystal representations using the machine learning model 106. The terms reducing, dimensionality reduction, projection, component analysis, feature space reduction, latent space engineering, feature space engineering, representation engineering, or latent space embedding can refer to a method of transforming a given input data with an initial number of dimensions to another form of data that has fewer dimensions than the initial number of dimensions. In some implementations, the terms can refer to the principle of reducing a set of input dimensions to a smaller set of output dimensions. In some implementations, the terms can refer to the principle of expanding a set of input dimensions to a set of output dimensions of a same or larger size.

The term normalizing can refer to a collection of methods for adjusting a dataset to align the dataset to a common scale. In some implementations, a normalizing method can include multiplying a portion or the entirety of a dataset by a factor. In some 8 implementations, a normalizing method can include adding or subtracting a constant from a portion or the entirety of a dataset. In some implementations, a normalizing method can include adjusting a portion or the entirety of a dataset to a known statistical distribution. In some implementations, a normalizing method can include adjusting a portion or the entirety of a dataset to a normal distribution. In some implementations, a normalizing method can include adjusting the dataset so that the signal strength of a portion or the entirety of a dataset is about the same.

Converting can include one or more steps of various conversions of data. In some implementations, converting can include normalizing data. In some implementations, converting can include performing a mathematical operation that computes a score based on a distance between two points in the data. In some implementations, the points in the data can include molecular or crystal representations. In some implementations, the distance can include a distance between two edges in a graph. In some implementations, the distance can include a distance between two nodes in a graph. In some implementations, the distance can include a distance between a node and an edge in a graph. In some implementations, the distance can include a Euclidean distance. In some implementations, the distance can include a non-Euclidean distance. In some implementations, the distance can be computed in a frequency space. In some implementations, the distance can be computed in Fourier space. In some implementations, the distance can be computed in Laplacian space. In some implementations, the distance can be computed in spectral space. In some implementations, the mathematical operation can be a monotonic function based on the distance. In some implementations, the mathematical operation can be a non-monotonic function based on the distance. In some implementations, the mathematical operation can be an exponential decay function. In some implementations, the mathematical operation can be a learned function.

In some implementations, converting can include transforming data in one representation to another representation. In some implementations, converting can include transforming data into another form of data with less dimensions. In some implementations, converting can include linearizing one or more curved paths in the data. In some implementations, converting can be performed on data comprising data in Euclidean space. In some implementations, converting can be performed on data comprising data in graph space. In some implementations, converting can be performed on data in a discrete space. In some implementations, converting can be performed on data comprising data in frequency space. In some implementations, converting can transform data in discrete space to continuous space, continuous space to discrete space, graph space to continuous space, continuous space to graph space, graph space to discrete space, discrete space to graph space, or any combination thereof. In some implementations, converting can include transforming data in discrete space into a frequency domain. In some implementations, converting can include transforming data in continuous space into a frequency domain. In some implementations, converting can include transforming data in graph space into a frequency domain.

In some implementations, reducing can include transforming a given input data with any initial number of dimensions to another form of data that has any number of dimensions fewer than the initial number of dimensions. In some implementations, reducing can include transforming input data into another form of data with fewer dimensions. In some implementations, reducing can include linearizing one or more curved paths in the input data to the output data. In some implementations, reducing can be performed on data comprising data in Euclidean space. In some implementations, reducing can be performed on data comprising data in graph space. In some implementations, reducing can be performed on data in a discrete space. In some implementations, reducing can transform data in discrete space to continuous space, continuous space to discrete space, graph space to continuous space, continuous space to graph space, graph space to discrete space, discrete space to graph space, or any combination thereof.

The terms clustering, cluster analysis, or generating modules can refer to a method of grouping samples in a dataset by some measure of similarity. Samples can be grouped in a set space, for example, element ‘a’ is in set ‘A’. Samples can be grouped in a continuous space, for example, element ‘a’ is a point in Euclidean space with distance ‘l’ away from the centroid of elements comprising cluster ‘A’. Samples can be grouped in a graph space, for example, element ‘a’ is highly connected to elements comprising cluster' A′. These terms can refer to the principle of organizing a plurality of elements into groups in some mathematical space based on some measure of similarity.

In some implementations, the method further includes clustering a cohort of molecular or crystal indications to determine one or more groups of molecular or crystal indications with similar structures, properties, or functions. Clustering can include grouping any number of samples in a dataset by any quantitative measure of similarity. In some implementations, clustering can include K-means clustering. In some implementations, clustering can include hierarchical clustering. In some implementations, clustering can include using random forest models. In some implementations, clustering can include boosted tree models. In some implementations, clustering can include using support vector machines. In some implementations, clustering can include calculating one or more N-1 dimensional surfaces in N-dimensional space that partitions a dataset into clusters. In some implementations, clustering can include distribution-based clustering. In some implementations, clustering can include fitting a plurality of prior distributions over the data distributed in N-dimensional space. In some implementations, clustering can include using density-based clustering. In some implementations, clustering can include using fuzzy clustering. In some implementations, clustering can include computing probability values of a data point belonging to a cluster. In some implementations, clustering can include using constraints. In some implementations, clustering can include using supervised learning. In some implementations, clustering can include using unsupervised learning.

In some implementations, clustering can include grouping samples based on similarity. In some implementations, clustering can include grouping samples based on quantitative similarity. In some implementations, clustering can include grouping samples based on one or more features of each sample. In some implementations, clustering can include grouping samples based on one or more labels of each sample. In some implementations, clustering can include grouping samples based on Euclidean coordinates. In some implementations, clustering can include grouping samples based on the features of the nodes and edges of each sample.

In some implementations, comparing can include comparing between a first group and a different second group. In some implementations, a first or a second group can each independently be a cluster. In some implementations, a first or a second group can each independently be a group of clusters. In some implementations, comparing can include comparing between one cluster with a group of clusters. In some implementations, comparing can include comparing between a first group of clusters with a second group of clusters different from the first group. In some implementations, one group can be one sample. In some implementations, one group can be a group of samples. In some implementations, comparing can include comparing between one sample versus a group of samples. In some implementations, comparing can include comparing between a group of samples versus a group of samples.

Comparing can include a variety of analytical methods carried out by a computer or a human. In some implementations, a statistical test can be carried out to identify one or more molecular or crystal representations that are the most different in one group versus a comparison group. In some implementations, clustering can be carried out on differences in molecular or crystal representations, which can lead to the identification of a set of molecular or crystal representations that show a high confidence for performing a satisfying a given machine learning task.

If the reliability metric for a given crystal structure calculated by the reliability metric calculator 104 is not within a predetermined threshold, the ground truth calculation engine 108 calculates a property metric for the crystal structure based one or more crystal structures in the set of crystal structures generated by the data ingestion engine 102. The ground truth calculation engine 108 can calculate a property metric for the crystal structure by using a ground truth calculation.

In some implementations, the ground truth calculation can be an ab initio calculation. In some implementations, the ground truth calculation can be based on interatomic interactions. The interatomic interactions can include potential energy functions. The potential energy functions can include one or more functions from OPLS, AMBER, CHARM, UFF, neural-network potential energy functions, or any combination thereof. In some implementations, the ground truth calculation can be based on electronic interactions. The ground truth calculation can be computed using a molecular dynamics or Monte Carlo method. The ground truth calculation can be computed using a quantum mechanical method.

The crystal structure indication reporter 110 receives a set of property metrics from either the machine learning model 106, the ground truth calculation engine 108, or both. The set of property metrics includes a property metric for each crystal structure in the set of crystal structures generated by the data ingestion engine 102. In some implementations, each property metric in the set of property metrics can have been generated at the same time. In some implementations, each property metric in the set of property metrics can have been generated one at a time.

The property metric for the crystal structure is received from the machine learning model 106 if the corresponding reliability metric for the crystal structure calculated by the reliability metric calculator 104 was within the predetermined threshold. The property metric for the crystal structure is received from the ground truth calculation engine 108 if the corresponding reliability metric for the crystal structure calculated by the reliability metric calculator 104 was not within the predetermined threshold. The crystal structure s reporter 110 can also receive the set of crystal structures associated with the received set of property metrics.

Upon receiving the set of property metrics and the set of crystal structures, the crystal structure indication reporter 110 can report a filtered set of crystal structure indications based on the set of property metrics and the set of crystal structures. In some implementations, the filtered set of crystal structure implementations can be a subset of the set of crystal structures generated by the data ingestion engine 102. The subset of the set of crystal structures can be generated by filtering the set of crystal structures based on the set of property metrics generated for the set of crystal structures.

In some implementations, the filtered set of crystal structure indications can include a set of polymorphic crystal structure indications. The set of polymorphic crystal structure indications can include stable and metastable polymorphic crystal structures of the one or more molecules. The set of polymorphic crystal structure indications can include synthesizable crystal structures of the one or more molecules. The set of polymorphic crystal structure indications can be sorted based on the set of property metrics to output a second report comprising a sorted set of polymorphic crystal structures.

In some implementations, the filtered set of crystal structure indications can include indications that one or more crystal structures among the set of crystal structures generated by the data ingestion engine 102 are likely to exist in an environment. In some implementations, the filtered set of crystal structure indications can include indications that one or more crystal structures among the set of crystal structures generated by the data ingestion engine 102 are likely to be good candidates for the development of a desired drug for pharmaceutical use. In some implementations, the filtered set of crystal structure indications can include indications that one or more crystal structures among the set of crystal structures generated by the data ingestion engine 102 are likely to be good candidates for the development of organic semiconductors.

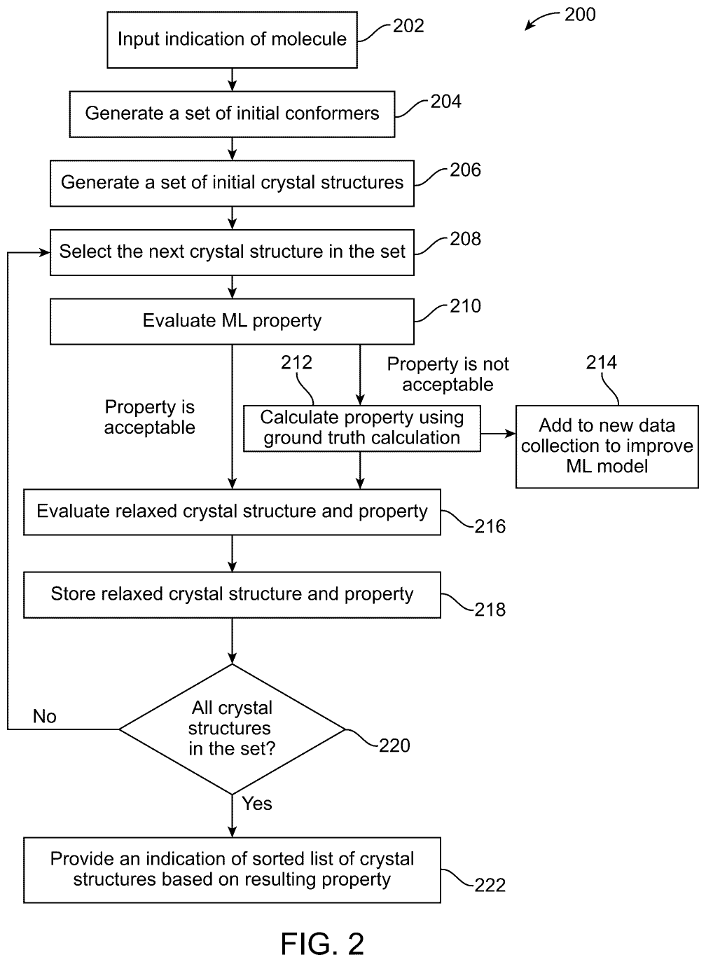

FIG. 2 is a flow diagram of an example process 200 for reporting a set of crystal structure indications for one or more molecules. For convenience, the process 200 will be described as being performed by a system of one or more computers located in one or more locations. For example, a CSP system, e.g., the CSP system 100 depicted in FIG. 1, appropriately programmed in accordance with this specification, can perform the process 200.

The system receives an input indication of a molecule (step 202). The indication can be information that represents the molecule. In particular, the indication can be an identifier of the molecule. Examples of indications that can be input into the system at step 202 are described above in reference to FIG. 1. The system can receive the indication from a user or from a machine learning model, as described above.

The system generates a set of initial conformers, based on the received indication (step 204). The system can generate one or more conformers in the set of conformers using cheminformatics tools or computational chemistry calculations or simulations, as described in further detail above in reference to FIG. 1. As part of generating the set of initial conformers, the system can also use computational chemistry calculations or simulations to optimize the geometry of one or more conformers in the set of initial conformers, as described in further detail above in reference to FIG. 1.

The system generates a set of initial crystal structures, using the set of initial conformers (step 206). The system can generate one or more crystal structures in the set of initial crystal structures by arranging one or more of the set of initial conformers in space. In particular, the system can optimize an arrangement of conformers in one or more crystal structures in the set of initial crystal structures, as described in further detail above in reference to FIG. 1.

The system selects a next crystal structure in the generated set of initial crystal structures (step 208). The selected next crystal structure is a crystal structure in the generated set of initial crystal structures that has not previously been selected by the system.

The system evaluates a machine learning (ML) property of the selected next crystal structure (step 210). The system can evaluate the ML property by generating a reliability metric of a machine learning algorithm for generating the ML property for the next crystal structure. In some implementations, the system can generate the reliability metric before, during, or after a generation of the corresponding ML property.

The reliability metric for the next crystal structure can be an indication of the accuracy of the machine learning algorithm in generating the ML property for the next crystal structure. In particular, the reliability metric falling within a predetermined threshold can be indicative of a high accuracy of the machine learning algorithm in generating the ML property for the next crystal structure. The reliability metric falling outside of the predetermined threshold can be indicative of a low accuracy of the machine learning algorithm in generating the ML property for the next crystal structure.

Upon evaluation of the ML property at step 210, the system can find that the ML property is either acceptable or not acceptable. The ML property can be acceptable if a reliability metric of a machine learning algorithm for generating the ML property falls within a predetermined threshold, as described above. The ML property can be not acceptable if the reliability metric falls outside the predetermined threshold, as described above.

If the ML property is not acceptable, the system calculates the property using a ground truth calculation (step 212). The property calculated using a ground truth calculation can correspond to the ML property that was evaluated at step 210. The ground truth calculation can be an ab initio calculation, can be based on interatomic interactions, and/or can be based on electronic interactions, as described in further detail above in reference to FIG. 1.

In some implementations, upon completing step 212, the system may optionally add the selected next crystal structure and its corresponding property calculated using the ground truth calculation to a new data collection that can be used to improve the machine learning algorithm used to generate the ML property (step 214). In particular, the selected next crystal structure and the calculated property can be stored in a training dataset. The training dataset can be prepared for training the machine learning model. The training dataset can be used to train the machine learning model. The training dataset can include at least 10, 100, 1 k, 10 k, 100 k, or 1M crystal structures and calculated properties. One or more machine learning model(s) can be trained using the training data set to perform one or more of: providing an indication of the one or molecules, generating a set of crystal structures based on the indication, generating a reliability metric for generating a property metric for each crystal structure in the set of crystal structures, and calculating the property metric for each crystal structure in the set of crystal structures to generate a set of property metrics by using a ground truth calculation of the crystal structure if the reliability metric for the crystal structure is not within a predetermined threshold.

If the ML property is acceptable, or if the ML property is not acceptable and upon completion of step 212 and optionally step 214, the system evaluates a relaxed crystal structure and property (step 216). In some implementations, evaluating a relaxed crystal structure and property includes reducing the energy of the selected next crystal structure to generate a crystal structure with reduced energy, and calculating a new property based on the crystal structure with reduced energy.

In some implementations, the system filters down the set of initial crystal structures based partially on the evaluated ML property, prior to evaluating a relaxed crystal structure and property.

The system stores the relaxed crystal structure and property (step 218). The system can store the relaxed crystal structure and property in a set of relaxed crystal structures and properties that is generated through repeated application of steps 208-218 to the set of initial crystal structures generated at step 206.

In some implementations, the system determines whether all crystal structures in the set of initial crystal structures have been selected (step 220). If not all crystal structures in the set of initial crystal structures have been selected, the system repeats steps 208-220. In particular, the system selects a next crystal structure in the set of initial crystal structures, where the next crystal structure is a crystal structure in the set of initial crystal structures which has not already been selected at step 208. Upon selecting the next crystal structure, the system performs steps 210-218 on the selected next crystal structure, as described above.

If all crystal structures in the set of initial crystal structures have been selected, the system provides an indication of a sorted list of crystal structures based on the property that was calculated for each crystal structure (step 222). The property for each crystal structure can be either of the ML property evaluated at step 210, if the ML property was acceptable, or the property calculated using a ground truth calculation at step 212, if the ML property was not acceptable.

The sorted list of crystal structures can be based on a set of crystal structure indications reported based on the set of relaxed crystal structures and properties. In some implementations, the set of crystal structure indications can include a set of polymorphic crystal structure indications, as described in further detail above in reference to FIG. 1. The set of polymorphic crystal structure indications can be sorted based on the set of relaxed crystal structures and properties to output a second report comprising a sorted set of polymorphic crystal structures. The sorted list of crystal structures can include the sorted set of polymorphic crystal structures.

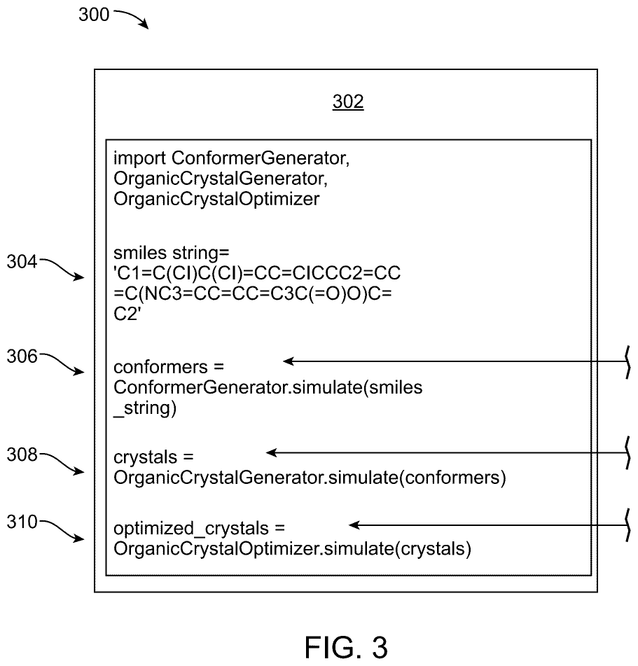

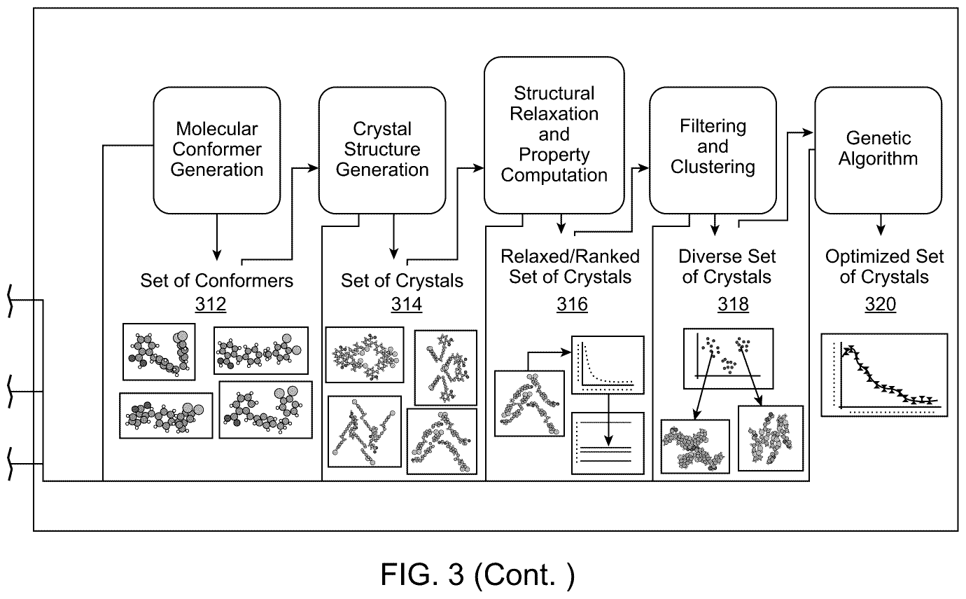

FIG. 3 is a diagram 300 illustrating views of example inputs and outputs of the steps in the process 200, described above in reference to FIG. 2, that can be implemented on a system of one or more computers located in one or more locations, such as the CSP system 100 depicted in FIG. 1. The panel 302 displays examples of input code that can cause the system to carry out various steps in the process 200.

In particular, the computer code 304 is an exemplary input indication of a molecule that is initially received by the system. In this example, the computer code 304 indicates the structural formula of the molecule, formatted as a SMILES string.

The computer code 306 is an exemplary command that can be entered into the system to cause the system to generate a set of initial conformers. In response to receiving the computer code 306, the system can generate a set of initial conformers. The set of conformers 312 is an example of the output that the system can generate upon performing the step of generating a set of initial conformers.

The computer code 308 is an exemplary command that can be entered into the system to cause the system to generate a set of initial crystal structures based on a set of initial conformers. In response to receiving the computer code 308, the system can generate a set of initial crystal structures. The set of crystals 314 is an example of the output that the system can generate upon performing the step of generating a set of initial crystal structures.

The computer code 310 is an exemplary command that can be entered into the system to cause the system to provide an indication of a sorted list of crystal structures based on a property that was calculated for each crystal structure in the set of initial crystal structures.

As described above in reference to FIG. 2, once the system has generated a set of initial crystal structures, the system generates a property for each crystal structure in the set of initial crystal structures and, in some implementations, uses the property to evaluate a relaxed crystal structure and property for each crystal structure in the set of initial crystal structures. The relaxed/ranked set of crystals 316 is an example of the output that the system can generate upon performing the steps of generating a property for each crystal structure in the set of initial crystal structures and using the property to evaluate a relaxed crystal structure and property for each crystal structure in the set of initial crystal structures.

As described above in reference to FIG. 2, in some implementations, once the system has evaluated a relaxed crystal structure and property for each crystal structure in the set of initial crystal structures, the system stores each relaxed crystal structure and property in a set of relaxed crystal structures and properties. The diverse set of crystals 318 is an example of the output that the system can generate to display the set of relaxed crystal structures and properties.

As described above in reference to FIG. 2, in some implementations, the system can use the set of relaxed crystal structures and properties to provide an indication of a sorted list of crystal structures based on the property that was calculated for each crystal structure. The optimized set of crystals 320 is an example of the output that the system can generate to display the sorted list of crystal structures.

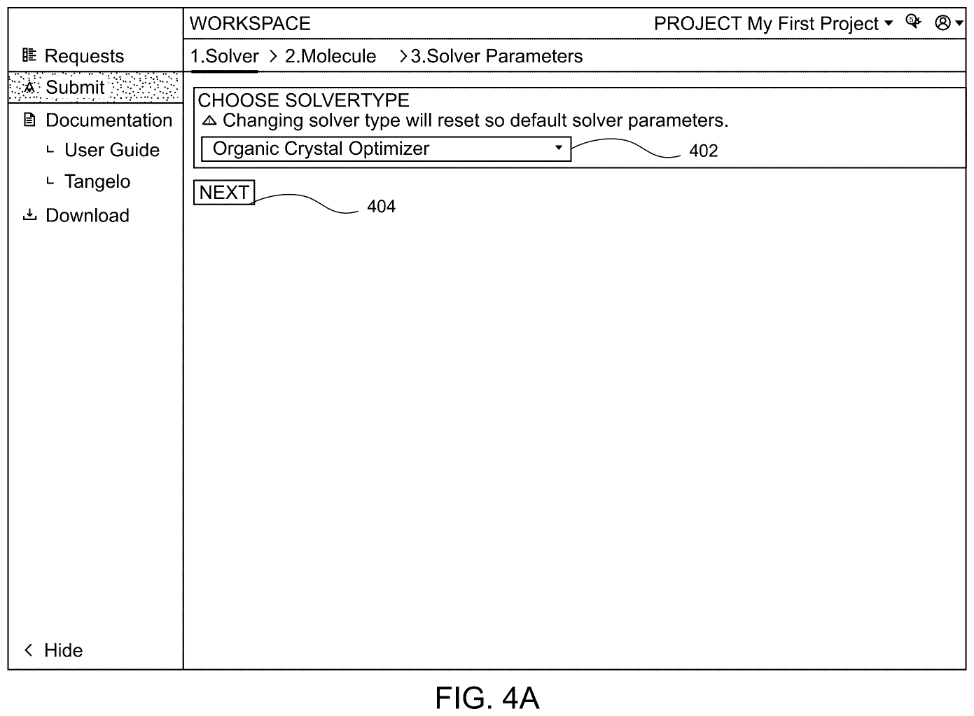

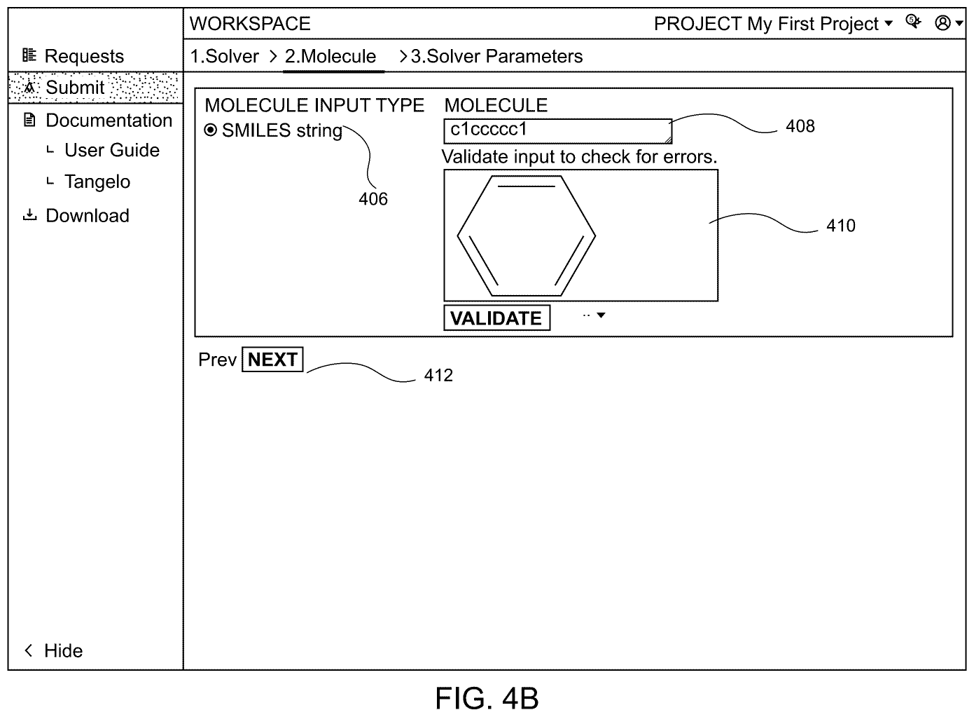

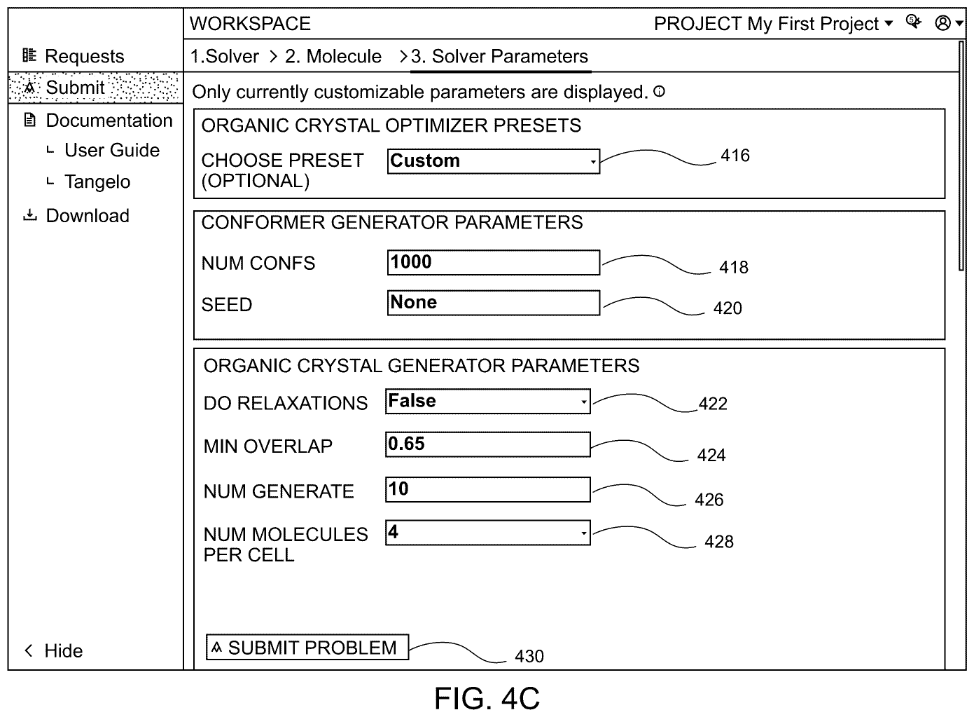

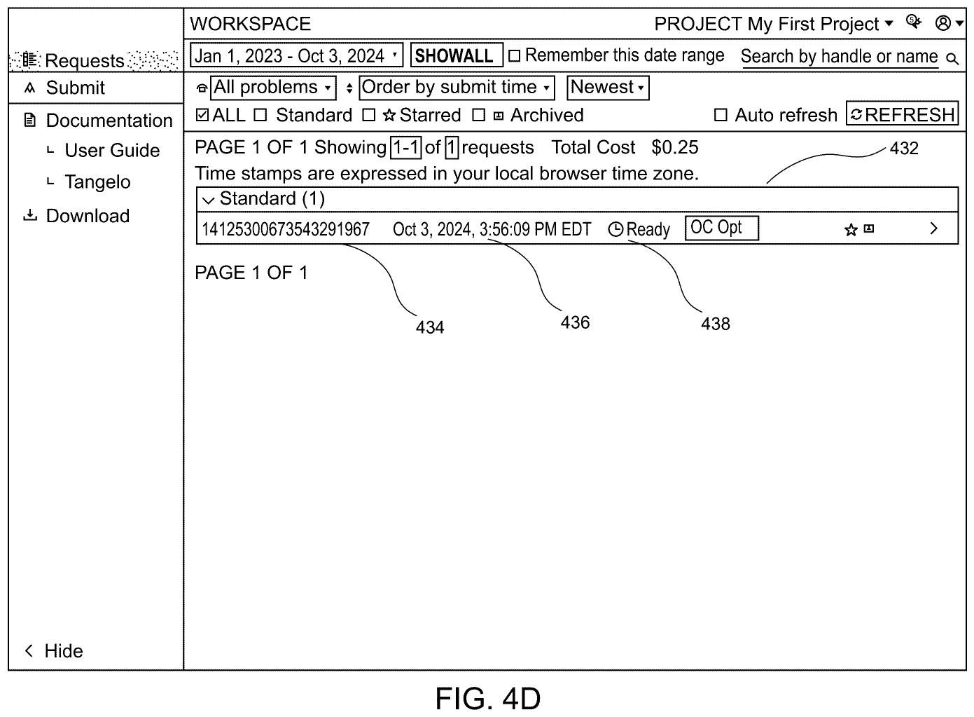

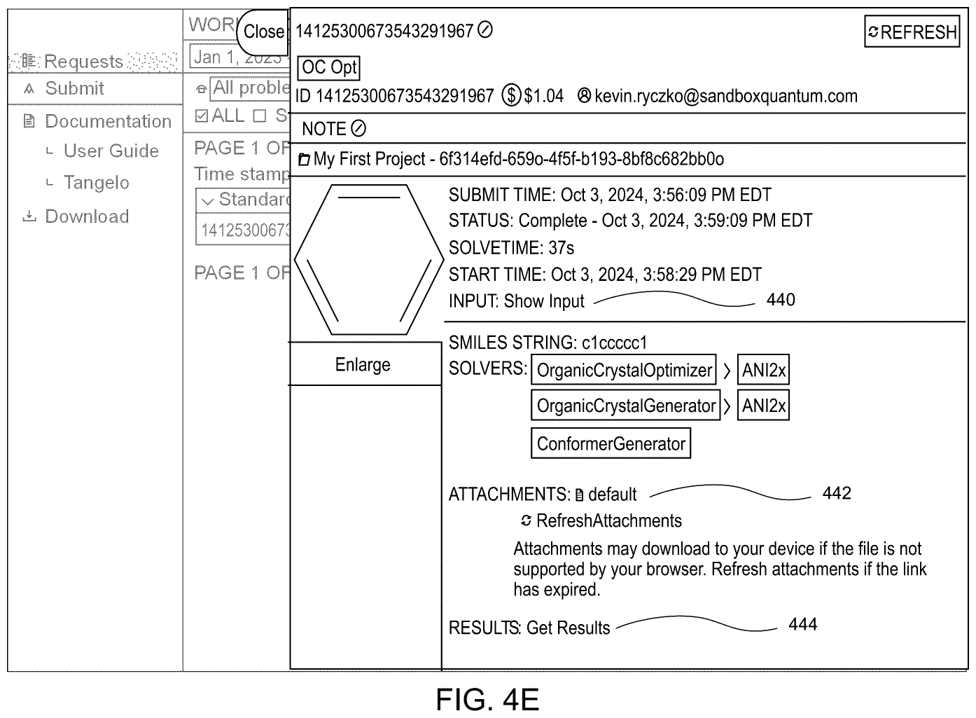

FIGS. 4A-4F are exemplary web-based graphical user interfaces with which a user can interact in order to cause a system, such as the CSP system 100 depicted in FIG. 1, to carry out the techniques described herein. In the exemplary user interfaces displayed in FIGS. 4A-4F, the user is using a cloud-based simulation platform that includes various computational chemistry techniques, to cause a computer program to perform various techniques described herein.

FIG. 4A is an exemplary interface that allows the user to select a computer program that will report a set of crystal structure indications for one or molecules. UI element 402 allows a user to select a solver type (e.g., which computer program to use) for generating and/or analyzing crystal structure candidates. Upon selecting a solver type, the user can use “Next” button 404 to navigate to the next interface.

FIG. 4B is an exemplary interface that allows a user to provide an indication of one or molecules. UI element 406 allows a user to select a molecule input type. The molecule input type can indicate a format in which the user will enter one or more indications of one or more molecules that the selected solver type will use to generate and/or analyze crystal structure candidates. In the example shown in FIG. 4B, the user has selected a molecule input type of a SMILES string.

Upon selecting a molecule input type, the user can input the molecule in the appropriate format into field 408. The interface can then display a visual indication corresponding to the input in field 410. Upon viewing the visual indication, the user can validate the input to check for errors by clicking the “Validate” button 412. Upon validating the input, the user can use the “Next” button 414 to navigate to the next interface.

FIG. 4C is an exemplary interface that allows a user to set various parameters indicating how the system is to apply the techniques described herein to the provided indication of the one or more molecules. In particular, UI element 416 allows the user to optionally choose a preset set of parameters in the case that the user does not wish to select their own custom set of parameters. The preset set of parameters can be stored in the system.

UI element 418 allows the user to select a number of conformers to be generated. UI element 420 allows the user to enter a seed crystal structure to be used by the system in generating and/or analyzing crystal structure candidates. UI element 422 allows the user to indicate whether the system is to perform relaxations on the generated set of crystal structures. Performing relaxations on the generated set of crystal structures can involve reducing the energy of the crystal structures in the set to generate a set of crystal structures with reduced energy.

UI element 424 allows the user to select a minimum overlap. The minimum overlap can be a minimum value of the overlap between atomic radii during random generation of the crystal structures. The amount of overlap between atomic radii can impact the random generation of the crystal structures. UI element 426 allows the user to select a number of crystal structures to be included in the generated set of crystal structures. UI element 428 allows the user to select a number of molecules per cell. The number of molecules per cell can be the number of copies in a cell of the molecule for which the crystal structure candidates are being generated and/or analyzed.

Upon selecting the parameters indicating how the system is to apply the techniques described herein to the provided indication of the one or more molecules, the user can use the “Submit Problem” button 430 to submit to the system a request to perform the process of generating and/or analyzing crystal structure candidates according to the selected parameters.

FIG. 4D is an exemplary interface that displays information related to the request submitted by the user to the system. In particular, UI element 432 displays an identification number for the request 434, a time/date stamp 436 indicating when the request was submitted, and a status of the request 438.

FIG. 4E is an exemplary interface that allows the user to view the results of the application of the techniques described herein by the computer program to the provided indication of the one or more molecules. The interface displays the submission data and time of the request corresponding to the results, the start and end times of the processing of the results, and the status of the request. The interface also displays the time taken by the system to produce the results, the input (in this case, a SMILES string) on which the results are based, the visual indication of the input, and the solvers that were used to produce the results. The “Show Input” link 440 allows the user to view the input on which the results are based. The “Get Results” link 444 allows the user to view the results. The “Attachments” link 442 allows the user to view any attachments that may accompany the results. Attachments can include one or more detailed outputs that each display one or more metrics associated with the generated crystal structure candidates.

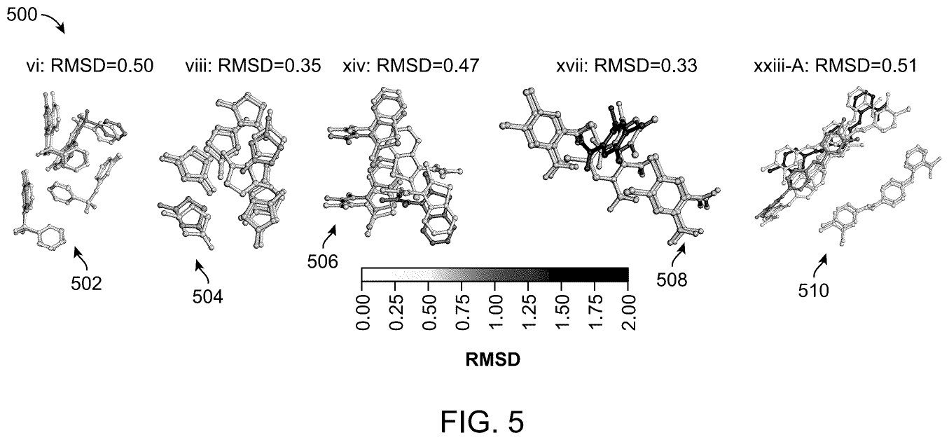

FIG. 5 is a set of output crystal structure indications 500 generated using the techniques described herein. Each of the molecule pairs 502-510 is the result of applying the techniques described herein to an input indication of a molecule. For each molecule pair, the experimental structure of the molecule is visualized as the unshaded molecule structure shown. The structure generated from an input indication of a molecule using the techniques described herein is fitted to the experimental structure, and shaded to show the root-mean-squared deviation (RMSD) of each individual molecule from the experimentally derived structure. Only molecules within the unit cell are visualized. Visualizations can be created using a Molecular Crystal Simulation Environment.

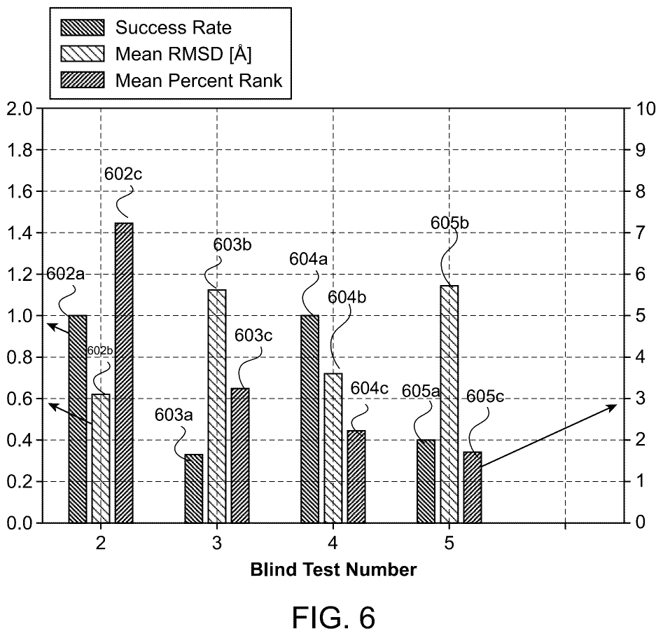

FIG. 6 is a graph illustrating the performance of the techniques described herein on a number of blind tests. For each blind test, a number of indications of a molecule were provided. For each indication of a molecule, the techniques described herein were applied to generate a sorted list of crystal structures for the corresponding molecule.

The x-axis (i.e., horizontal axis) of the graph represents the numerical label of the blind test that was performed. The y-axis (i.e., vertical axis) on the left side of the graph represents the success rate or the mean RMSD (in angstrom (Å)) achieved by the techniques described herein on a given blind test. The success rate is the rate at which the techniques described herein generated a match for a given blind test. Generating a match means generating a crystal structure indication that is within 0.8 Å RMSD from the ground-truth structure, e.g. a structure experimentally derived.

The y-axis (i.e., vertical axis) on the right side of the graph represents the mean percent rank achieved by the techniques described herein on a given blind test. The mean percent rank corresponds to the average rank that a match was assigned in the sorted list of crystal structures generated by the techniques described herein in the given blind test.

In particular, for each blind test indicated on the x-axis, the left-most bar (e.g., bars 602a-605a) represents the success rate (with a value corresponding to its height measured on the left vertical axis) achieved by the techniques described herein in the blind test. The center bar (e.g., bars 602b-605b) represents the mean RMSD (with a value in Å corresponding to its height measured on the left vertical axis) achieved by the techniques described herein in the blind test. The right-most bar (e.g., bars 602c-605c) represents the mean percent rank (with a value corresponding to its height measured on the right vertical axis) achieved by the techniques described herein in the blind test.

Of the molecules in the blind tests for which matches were generated, the matches were ranked on average in the top 4.7% by the techniques described herein. For eight of the molecules for which an indication was provided, the matches were ranked within the top 4%, and for 3 of those eight molecules, the matches were ranked within the top 1%.

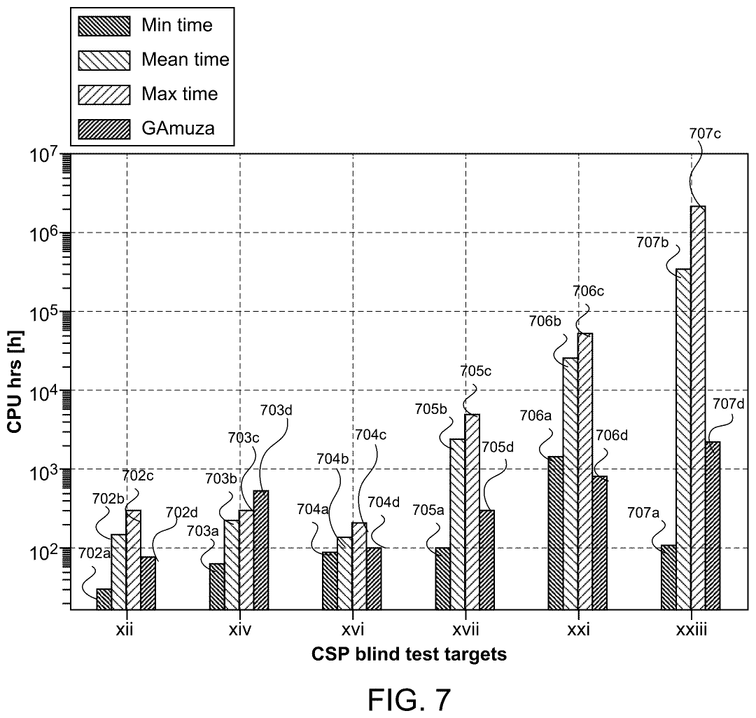

FIG. 7 is a graph illustrating the time taken to generate predicted structures of various target molecules using the techniques described herein to generate sets of crystal structure indications, as compared to the time taken to generate the predicted structures by alternative methods. The x-axis (i.e., horizontal axis) of the graph represents the label (in roman numerals) of a particular CSP blind test target molecule. The y-axis (i.e., vertical axis) of the graph represents the number of CPU hours, plotted on a logarithmic scale, taken to generate a predicted structure of a given target molecule.

For each target molecule indicated on the x-axis, the left-most bar (e.g., bars 702a-707a) represents the minimum time taken out of all the methods to generate the predicted structure for the target molecule. The second bar from the left (e.g., bars 702b-707b) represents the mean time taken over all the methods to generate the predicted structure for the target molecule. The third bar from the left (e.g., bars 702c-707c) represents the maximum time taken out of all the methods to generate the predicted structure for the target molecule. Finally, the right-most bar (e.g., bars 702d-707d) represents the time taken by the techniques described herein to generate the predicted structure for the target molecule.

For four out of the six target molecules, the techniques described herein achieved a faster run time that the mean run time. For one of the target molecules (xxi), the techniques described herein achieved a faster run time than the fastest method.

In particular, the graph in FIG. 7 reflects an advantage of the techniques described herein, in that they approach speeds similar to methods employing force-fields for ranking, but are orders of magnitude faster than methods using ab initio methods. Additionally, the aspects of the techniques described herein that incorporate machine learning for ranking in CSP allow for speeds almost as fast as force-field-based methods, while also approaching the accuracy achieved by quantum chemistry methods.

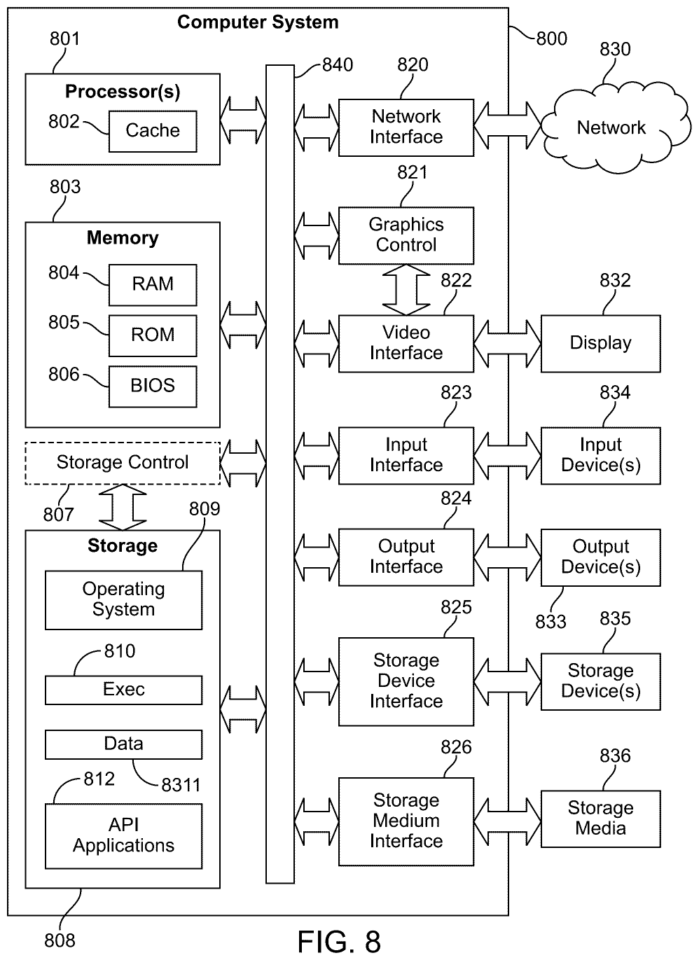

FIG. 8 is a block diagram depicting an exemplary machine that includes a computer system 800 (e.g., a processing or computing system) within which a set of instructions can execute for causing a device to perform or execute any one or more of the implementations and/or methodologies for generating conformers, generating an arrangement of conformers, generating a crystal structure, optimizing the geometry of crystal structures, generating an output of crystal structure indications, performing active learning, training a CSP model machine learning algorithm, or any combination thereof. The components in FIG. 8 are examples only and do not limit the scope of use or functionality of any hardware, software, embedded logic component, or a combination of two or more such components implementing particular implementations.

Computer system 800 may include one or more processors 801, a memory 803, and a storage 808 that communicate with each other, and with other components, via a bus 840. The bus 840 may also link a display 838, one or more input devices 833 (which may, for example, include a keypad, a keyboard, a mouse, a stylus, etc.), one or more output devices 834, one or more storage devices 835, and various tangible storage media 836. All of these elements may interface directly or via one or more interfaces or adaptors to the bus 840. For instance, the various tangible storage media 836 can interface with the bus 840 via storage medium interface 886. Computer system 800 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers. The computer system 800 can be configured to generate a set of conformers one at a time or at the same time. The computer system 800 can be configured to generate a set of crystal structures of the one or more molecules one at a time, at the same time, or in parallel. The computer system 800 can be configured to generate a set of crystal structures in parallel using one or more distributed processes, which can be coordinated through, e.g., MPI. The computer system 800 can be configured to generate a reliability metric for each crystal structure in the set of crystal structures at the same time, or one at a time. The computer system 800 can be configured to generate a property metric for each crystal structure in the set of crystal structures at the same time, or one at a time.

Computer system 800 includes one or more processor(s) 801 (e.g., central processing units (CPUs), general purpose graphics processing units (GPGPUs), or quantum processing units (QPUs)) that carry out functions. Computer system 800 may be one of various high performance computing platforms. For instance, the one or more processor(s) 801 may form a high performance computing cluster. In some implementations, the one or more processors 801 may form a distributed computing system connected by wired and/or wireless networks. In some implementations, arrays of CPUs, GPUs, QPUs, or any combination thereof may be operably linked to implement any one of the methods disclosed herein. Processor(s) 801 optionally contains a cache memory unit 808 for temporary local storage of instructions, data, or computer addresses. Processor(s) 801 are configured to assist in execution of computer readable instructions. Computer system 800 may provide functionality for the components depicted in FIG. 8 as a result of the processor(s) 801 executing non-transitory, processor-executable instructions embodied in one or more tangible computer-readable storage media, such as memory 803, storage 808, storage devices 835, and/or storage medium 836. The computer-readable media may store software that implements particular implementations, and processor(s) 801 may execute the software. Memory 803 may read the software from one or more other computer-readable media (such as mass storage device(s) 835, 836) or from one or more other sources through a suitable interface, such as network interface 880. The software may cause processor(s) 801 to carry out one or more processes or one or more steps of one or more processes described or illustrated herein. Carrying out such processes or steps may include defining data structures stored in memory 803 and modifying the data structures as directed by the software.

The memory 803 may include various components (e.g., machine readable media) including, but not limited to, a random access memory component (e.g., RAM 804) (e.g., static RAM (SRAM), dynamic RAM (DRAM), ferroelectric random access memory (FRAM), phase-change random access memory (PRAM), etc.), a read-only memory component (e.g., ROM 805), and any combinations thereof. ROM 805 may act to communicate data and instructions unidirectionally to processor(s) 801, and RAM 804 may act to communicate data and instructions bidirectionally with processor(s) 801. ROM 805 and RAM 804 may include any suitable tangible computer-readable media described below. In one example, a basic input/output system 806 (BIOS), including basic routines that help to transfer information between elements within computer system 800, such as during start-up, may be stored in the memory 803. 2

Fixed storage 808 is connected bidirectionally to processor(s) 801, optionally through storage control unit 807. Fixed storage 808 provides additional data storage capacity and may also include any suitable tangible computer-readable media described herein. Storage 808 may be used to store operating system 809, executable(s) 810, data 811, applications 818 (application programs), and the like. Storage 808 can also include an optical disk drive, a solid-state memory device (e.g., flash-based systems), or a combination of any of the above. Information in storage 808 may, in appropriate cases, be incorporated as virtual memory in memory 803.

In one example, storage device(s) 835 may be removably interfaced with computer system 800 (e.g., via an external port connector (not shown)) via a storage device interface 885. Particularly, storage device(s) 835 and an associated machine-readable medium may provide non-volatile and/or volatile storage of machine-readable instructions, data structures, program modules, and/or other data for the computer system 800. In one example, software may reside, completely or partially, within a machine-readable medium on storage device(s) 835. In another example, software may reside, completely or partially, within processor(s) 801.

Bus 840 connects a wide variety of subsystems. Herein, reference to a bus may encompass one or more digital signal lines serving a common function, where appropriate. Bus 840 may be any of several types of bus structures including, but not limited to, a memory bus, a memory controller, a peripheral bus, a local bus, and any combinations thereof, using any of a variety of bus architectures. As an example, and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 800 may also include an input device 833. In one example, a user of computer system 800 may enter commands and/or other information into computer system 800 via input device(s) 833. Examples of an input device(s) 833 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some implementations, the input device is a Kinect, Leap Motion, or the like. Input device(s) 833 may be interfaced to bus 840 via any of a variety of input interfaces 883 (e.g., input interface 883)

including, but not limited to, serial, parallel, game port, USB, FIREWIRE, THUNDERBOLT, or any combination of the above. In some implementations, an input device 833 may be used to generate conformers, generate an arrangement of conformers, generate a crystal structure, optimize the geometry of crystal structures, generate an output of crystal structure indications, perform active learning, train a CSP model machine learning algorithm, or any combination thereof. In some implementations, generating conformers, generating an arrangement of conformers, generating a crystal structure, optimizing the geometry of crystal structures, generating an output of crystal structure indications, performing active learning, training a CSP model machine learning algorithm, or any combination thereof may be performed using human inputs through an input device 833.

In particular implementations, when computer system 800 is connected to network 830, computer system 800 may communicate with other devices, specifically mobile devices and enterprise systems, distributed computing systems, cloud storage systems, cloud computing systems, and the like, connected to network 830. Communications to and from computer system 800 may be sent through network interface 880. For example, network interface 880 may receive incoming communications (such as requests or responses from other devices) in the form of one or more packets (such as Internet Protocol (IP) packets) from network 830, and computer system 800 may store the incoming communications in memory 803 for processing. Computer system 800 may similarly store outgoing communications (such as requests or responses to other devices) in the form of one or more packets in memory 803 and communicated to network 830 from network interface 880. Processor(s) 801 may access these communication packets stored in memory 803 for processing.

Examples of the network interface 880 include, but are not limited to, a network interface card, a modem, and any combination thereof. Examples of a network 830 or network segment 830 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 830, may employ a wired and/or a wireless mode of communication. In general, any network topology may be used.

Information and data can be displayed through a display 838. Examples of a display 838 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 838 can interface to the processor(s) 801, memory 803, and fixed storage 808, as well as other devices, such as input device(s) 833, via the bus 840. The display 838 is linked to the bus 840 via a video interface 888, and transport of data between the display 838 and the bus 840 can be controlled via the graphics control 881. In some implementations, the display is a video projector. In some implementations, the display is a head-mounted display (HMD) such as a VR headset. In further implementations, suitable VR headsets include, by way of non-limiting examples, HTC Vive, Oculus Rift, Samsung Gear VR, Microsoft HoloLens, Razer OSVR, FOYE VR, Zeiss VR One, Avegant Glyph, Freefly VR headset, and the like. In still further implementations, the display is a combination of devices such as those disclosed herein.

In addition to a display 838, computer system 800 may include one or more other peripheral output devices 834 including, but not limited to, an audio speaker, a printer, a storage device, and any combinations thereof. Such peripheral output devices may be connected to the bus 840 via an output interface 884. Examples of an output interface 884 include, but are not limited to, a serial port, a parallel connection, a USB port, a FIREWIRE port, a THUNDERBOLT port, and any combinations thereof.

In addition, or as an alternative, computer system 800 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the implementations disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the implementations disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the implementations disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, and tablet computers.

In some implementations, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some implementations, the operating system is provided by cloud computing. Those of skill in the art will also recognize that suitable mobile smartphone operating systems include, by way of non-limiting examples, Nokia® Symbian® OS, Apple® Ios®, Research In Motion® BlackBerry OS®, Google® Android®, Microsoft® Windows Phone® OS, Microsoft® Windows Mobile® OS, Linux®, and Palm® WebOS®.

In some implementations, a computer system 800 may be accessible through a user terminal to receive user commands. The user commands may include line commands, scripts, programs, etc., and various instructions executable by the computer system 800. A computer system 800 may receive instructions to generate conformers, generate an arrangement of conformers, generate a crystal structure, optimize the geometry of crystal structures, generate an output of crystal structure indications, perform active learning, train a CSP model machine learning algorithm, or any combination thereof, or schedule a computing job for the computer system 800 to carry out any instructions.

In some implementations, the present disclosure describes a non-transitory computer-readable storage media encoded with a computer program including instructions executable by one or more processors to generate conformers, generate an arrangement of conformers, generate a crystal structure, optimize the geometry of crystal structures, generate an output of crystal structure indications, perform active learning, train a CSP model machine learning algorithm, or any combination thereof using any one of the methods disclosed herein. In some implementations, a non-transitory computer-readable storage media may comprise instructions for generating conformers, generating an arrangement of conformers, generating a crystal structure, optimizing the geometry of crystal structures, generating an output of crystal structure indications, performing active learning, training a CSP model machine learning algorithm, or any combination thereof. In some implementations, the platforms, systems, media, and methods disclosed herein include one or more non-transitory computer readable storage media encoded with a program including instructions executable by the operating system of an optionally networked computing device.

In further implementations, a computer readable storage medium is a tangible component of a computing device. In still further implementations, a computer readable storage medium is optionally removable from a computing device. In some implementations, a computer readable storage medium includes, by way of non-limiting examples, flash memory devices, solid state memory, magnetic disk drives, magnetic tape drives, optical disk drives, distributed computing systems including cloud computing systems and services, and the like. In some implementations, the program and instructions are permanently, substantially permanently, semi-permanently, or non-transitorily encoded on the media.

In some implementations, the present disclosure describes a computer program product comprising a computer-readable medium having computer-executable code encoded therein, the computer-executable code adapted to be executed to implement any one of the methods disclosed herein. In some implementations, the platforms, systems, media, and methods disclosed herein include at least one computer program, or use of the same.

A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages. In some implementations, APIs may comprise various languages, for example, languages in various releases of TensorFlow, Theano, Keras, PyTorch, or any combination thereof which may be implemented in various releases of Python, Python3, C, C#, C++, MatLab, R, Java, or any combination thereof.

The functionality of the computer readable instructions may be combined or distributed as desired in various environments. In some implementations, a computer program comprises one sequence of instructions. In some implementations, a computer program comprises a plurality of sequences of instructions. In some implementations, a computer program is provided from one location. In other implementations, a computer program is provided from a plurality of locations. In various implementations, a computer program includes one or more software modules. In various implementations, a computer program includes, in part or in whole, one or more web applications, one or more standalone applications, one or more web browser plug-ins, extensions, add-ins, or add-ons, or combinations thereof.

In some implementations, a computer program includes a web application. In some implementations, a user may enter a query for generating conformers, generating an arrangement of conformers, generating a crystal structure, optimizing the geometry of crystal structures, generating an output of crystal structure indications, performing active learning, training a CSP model machine learning algorithm, or any combination thereof through a web application. In some implementations, a user may generate conformers, generate an arrangement of conformers, generate a crystal structure, optimize the geometry of crystal structures, generate an output of crystal structure indications, perform active learning, train a CSP model machine learning algorithm, or any combination thereof through a web application. In light of the disclosure provided herein, those of skill in the art will recognize that a web application, in various implementations, utilizes one or more software frameworks and one or more database systems. In some implementations, a web application is created upon a software framework such as Microsoft® .NET or Ruby on Rails (RoR). In some implementations, a web application utilizes one or more database systems including, by way of non-limiting examples, relational, non-relational, object oriented, associative, XML, and document oriented database systems. In further implementations, suitable relational database systems include, by way of non-limiting examples, Microsoft® SQL Server, mySQL™, and Oracle®. Those of skill in the art will also recognize that a web application, in various implementations, is written in one or more versions of one or more languages. A web application may be written in one or more markup languages, presentation definition languages, client-side scripting languages, server-side coding languages, database query languages, or combinations thereof. In some implementations, a web application is written to some extent in a markup language such as Hypertext Markup Language (HTML), Extensible Hypertext Markup Language (XHTML), or extensible Markup Language (XML). In some implementations, a web application is written to some extent in a presentation definition language such as Cascading Style Sheets (CSS). In some implementations, a web application is written to some extent in a client-side scripting language such as Asynchronous JavaScript and XML (AJAX), Flash® ActionScript, JavaScript, or Silverlight®. In some implementations, a web application is written to some extent in a server-side coding language such as Active Server Pages (ASP), ColdFusion®, Perl, Java™, JavaServer Pages (JSP), Hypertext Preprocessor (PHP), Python™, Ruby, Tel, Smalltalk, WebDNA®, or Groovy. In some implementations, a web application is written to some extent in a database query language such as Structured Query Language (SQL). In some implementations, a web application integrates enterprise server products such as IBM® Lotus Domino®.

In some implementations, a computer program includes a mobile application provided to a mobile computing device. In some implementations, the mobile application is provided to a mobile computing device at the time it is manufactured. In other implementations, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB .NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform.

Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (Ios) SDK, Android™ SDK, BlackBerry® SDK, BREW SDK, Palm® OS SDK, Symbian SDK, webOS SDK, and Windows® Mobile SDK.

In some implementations, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some implementations, a computer program includes one or more executable complied applications.

In some implementations, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various implementations, a software module comprises a file, a section of code, a programming object, a programming structure, a distributed computing resource, a cloud computing resource, or combinations thereof. In further various implementations, a software module comprises a plurality of files, a plurality of sections of code, a plurality of programming objects, a plurality of programming structures, a plurality of distributed computing resources, a plurality of cloud computing resources, or combinations thereof. In various implementations, the one or more software modules comprise, by way of non-limiting examples, a web application, a mobile application, a standalone application, and a distributed or cloud computing application. In some implementations, software modules are in one computer program or application. In other implementations, software modules are in more than one computer program or application. In some implementations, software modules are hosted on one machine. In other implementations, software modules are hosted on more than one machine. In further implementations, software modules are hosted on a distributed computing platform such as a cloud computing platform. In some implementations, software modules are hosted on one or more machines in one location. In other implementations, software modules are hosted on one or more machines in more than one location.

In some implementations, the platforms, systems, media, and methods disclosed herein include one or more databases, or use of the same. In view of the disclosure provided herein, those of skill in the art will recognize that many databases are suitable for storage and retrieval of information about generating conformers, generating an arrangement of conformers, generating a crystal structure, optimizing the geometry of crystal structures, generating an output of crystal structure indications, performing active learning, training a CSP model machine learning algorithm, or any combination thereof. In various implementations, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, Atomic Simulation Environment Database, and MongoDB. In some implementations, a database is Internet-based. In further implementations, a database is web-based. In still further implementations, a database is cloud computing-based. In a particular implementation, a database is a distributed database. In other implementations, a database is based on one or more local computer storage devices.

In some cases, the systems and methods disclosed herein may be performed with the aid of a quantum computing system. In some cases, a computer-implemented method of the present disclosure may be performed at least partially by a quantum computer. In some cases, a computing system of the present disclosure may comprise a hybrid computing unit. In some cases, a hybrid computing unit may comprise a classical computer and quantum computer. The quantum computer may be configured to perform one or more quantum algorithms for solving a computational problem (e.g., at least a portion of a quantum chemistry simulation).

The one or more quantum algorithms may be executed using a quantum computer, a quantum-ready computing service, or a quantum-enabled computing service. For instance, the one or more quantum algorithms may be executed using the systems or methods described in U.S. Patent Publication No. 2018/0107526, entitled “METHODS AND SYSTEMS FOR QUANTUM READY AND QUANTUM ENABLED COMPUTATIONS”, which is entirely incorporated herein by reference. The classical computer may comprise at least one classical processor and computer memory and may be configured to perform one or more classical algorithms for solving a computational problem (e.g., at least a portion of a quantum chemistry simulation).

The digital computer may comprise at least one computer processor and computer memory, wherein the digital computer may include a computer program with instructions executable by the at least one computer processor to render an application. The application may facilitate use of the quantum computer and/or the classical computer by a user.

Some implementations may use quantum computers along with classical computers operating on bits, such as personal desktops, laptops, supercomputers, distributed computing, clusters, cloud-based computing resources, smartphones, or tablets.

The system may comprise an interface for a user. In some cases, the interface may comprise an application programming interface (API). The interface may provide a programmatic model that abstracts away (e.g., by hiding from the user) the internal details (e.g., architecture and operations) of the quantum computer. In some cases, the interface may minimize a need to update the application programs in response to changing quantum hardware. In some cases, the interface may remain unchanged when the quantum computer has a change in internal structure.

The present disclosure provides systems and methods that may include non-classical (e.g., quantum) computing or use of non-classical (e.g., quantum) computing. Quantum computers may be able to solve certain classes of computational tasks more efficiently than classical computers. However, quantum computation resources may be rare and expensive, and may involve a certain level of expertise to be used efficiently or effectively (e.g., cost-efficiently or cost-effectively). A number of parameters may be tuned in order for a quantum computer to deliver its potential computational power.

Quantum computers (or other types of non-classical computers) may be able to work alongside classical computers as co-processors. A hybrid architecture (e.g., computing system) comprising a classical computer and a quantum computer can be very efficient for addressing complex computational tasks, such as computational chemistry calculations and simulations. Systems and methods disclosed herein may be able to efficiently and accurately or break down a quantum chemistry problem and delegate appropriate components of the quantum chemistry simulations to the quantum computer or the classical computer. Systems and methods disclosed herein may be able to efficiently and accurately train a machine learning algorithm using a quantum computer.

Although the present disclosure has referred to quantum computers in some implementations, methods and systems of the present disclosure may be employed for use with other types of computers, which may be non-classical computers. Such non-classical computers may comprise quantum computers, hybrid quantum computers, quantum-type computers, or other computers that are not classical computers. Examples of non-classical computers may include, but are not limited to, Hitachi Ising solvers, coherent Ising machines based on optical parameters, and other solvers which utilize different physical phenomena to obtain more efficiency in solving particular classes of problems.

In some cases, a quantum computer may comprise one or more adiabatic quantum computers, quantum gate arrays, one-way quantum computers, topological quantum computers, quantum Turing machines, superconductor-based quantum computers, trapped ion quantum computers, trapped atom quantum computers, optical lattices, quantum dot computers, spin-based quantum computers, spatial-based quantum computers, Loss-Di Vincenzo quantum computers, nuclear magnetic resonance (NMR) based quantum computers, solution-state NMR quantum computers, solid-state NMR quantum computers, solid-state NMR Kane quantum computers, electrons-on-helium quantum computers, cavity-quantum-electrodynamics based quantum computers, molecular magnet quantum computers, fullerene-based quantum computers, linear optical quantum computers, diamond-based quantum computers, nitrogen vacancy (NV) diamond-based quantum computers, Bose-Einstein condensate-based quantum computers, transistor-based quantum computers, and rare-earth-metal-ion-doped inorganic crystal based quantum computers. A quantum computer may comprise one or more of: quantum annealers, Ising solvers, optical parametric oscillators (OPO), and gate models of quantum computing.

In some cases, a non-classical computer of the present disclosure may comprise a noisy intermediate-scale quantum device. “Noisy” may imply that incomplete control over the qubits is present and the “Intermediate-Scale” may refer to the number of qubits which may range from 50 to a few hundreds. Several physical systems made from superconducting qubits, artificial atoms, ion traps are proposed so far as feasible candidates to build NISQ quantum device and ultimately universal quantum computers.

In some cases, a classical simulator of the quantum circuit can be used which can run on a classical computer like a MacBook Pro laptop, a Windows laptop, or a Linux laptop. In some cases, the classical simulator can run on a cloud computing platform having access to multiple computing nodes in a parallel or distributed manner. In some cases, all or a portion of a quantum mechanical energy and/or electronic structure calculation may be performed using the classical simulator.

The methods described herein may be performed on an analogue quantum simulator. An analogue quantum simulator may be a quantum mechanical system consisting of a plurality of manufactured qubits. An analogue quantum simulator may be designed to simulate quantum systems by using physically different but mathematically equivalent or approximately equivalent systems. In an analogue quantum simulator, each qubit may be realized in an ion of strings of trapped atomic ions in linear radiofrequency traps. To each qubit may be coupled a source of bias called a local field bias. The local field biases on the qubits may be programmable and controllable. In some cases, a qubit control system comprising a digital processing unit is connected to the system of qubits and is capable of programming and tuning the local field biases on the qubits.

While preferred implementations of the present disclosure have been shown and described herein, it will be obvious to those skilled in the art that such implementations are provided by way of example only. Numerous variations, changes, and substitutions will now occur to those skilled in the art without departing from the disclosure. It should be understood that various alternatives to the implementations of the present disclosure may be employed in practicing the present disclosure. It is intended that the following claims define the scope of the present disclosure and that methods and structures within the scope of these claims and their equivalents be covered thereby.

Implementations of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Implementations of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone, running a messaging application, and receiving responsive messages from the user in return.

An electronic document, which for brevity will simply be referred to as a document, may, but need not, correspond to a file. A document may be stored in a portion of a file that holds other documents, in a single file dedicated to the document in question, or in multiple coordinated files.

In this specification, the term “database” will be used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” will be used broadly to refer to a software based system or subsystem that can perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

While this specification contains many specific implementation details, these should not be construed as limitations on the scope of any invention or on the scope of what may be claimed, but rather as descriptions of features that may be specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the implementations described above should not be understood as requiring such separation in all implementations, and it should be understood that the described program components and systems can generally be integrated together in a single software product or packaged into multiple software products.

Particular implementations of the subject matter have been described. Other implementations are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A computer-implemented method for reporting a set of crystal structure indications for one or more molecules, comprising:
providing an indication of the one or more molecules;
generating a set of crystal structures based on the indication;
generating a reliability metric of a machine learning model for generating a property metric for each crystal structure in the set of crystal structures;
calculating the property metric for each crystal structure in the set of crystal structures to generate a set of property metrics, by (i) using the machine learning model if the reliability metric for the crystal structure is within a predetermined threshold, and (ii) using a ground truth calculation of the crystal structure if the reliability metric for the crystal structure is not within the predetermined threshold; and
taking an action based on the set of crystal structure indications for the one or more molecules, wherein the set of crystal structure indications is based on the set of crystal structures and the set of property metrics.

2. The computer-implemented method of claim 1, wherein the property comprises density, solubility, stability, ADMET, or any combination thereof.

3. The computer-implemented method of claim 1, wherein the property metric is an energy metric.

4. The computer-implemented method of claim 3, wherein the energy metric is potential energy.

5. The computer-implemented method of claim 3, wherein the energy metric is free energy.

6. The computer-implemented method of claim 1, wherein the machine learning model comprises a neural network.

7. The computer-implemented method of claim 1, wherein the reliability metric is indicative of an accuracy of the machine learning algorithm for generating the property metric.

8. The computer-implemented method of claim 1, wherein calculating the property metric for each crystal structure in the set of crystal structures to generate a set of property metrics further comprises, if the reliability metric for the crystal structure is not within the predetermined threshold, storing the crystal structure and the calculated property metric in a training dataset.

9. The computer-implemented method of claim 8, further comprising preparing the training dataset for training the machine learning model.

10. The computer-implemented method of claim 9, further comprising training the machine learning model using the training dataset.

11. The computer-implemented method of claim 8, wherein the training dataset comprises at least 10, 100, 1 k, 10 k, 100 k, or 1M crystal structures and calculated energy metrics.

12. The computer-implemented method of claim 1, wherein the set of crystal structure indications comprises a set of polymorphic crystal structure indications, and wherein the method further comprises: sorting the set of polymorphic crystal structure indications based on the set of property metrics to output a report comprising a sorted set of polymorphic crystal structures.

13. The computer-implemented method of claim 1, wherein the ground truth calculation is based on interatomic interactions.

14. The computer-implemented method of claim 13, wherein the interatomic interactions comprise potential energy functions.

15. The computer-implemented method of claim 14, wherein the potential energy functions comprise one or more functions from OPLS, AMBER, CHARM, UFF, neural-network potential energy functions, or any combination thereof.

16. The computer-implemented method of claim 13, wherein the ground truth calculation is based on electronic interactions.

17. The computer-implemented method of claim 13, wherein the ground truth calculation is computed using any one of a molecular dynamics method, a Monte Carlo method, or a quantum mechanical method.

18. The computer-implemented method of claim 1, wherein taking an action comprises forwarding data characterizing the set of crystal structure indications for display.

19. The computer-implemented method of claim 1, wherein providing an indication of the one or more molecules comprises receiving an indication from a generative machine learning model.

20. A computer-implemented active learning method for reporting a set of crystal structure indications for one or more molecules, comprising:
providing an indication of the one or more molecules;
generating a set of crystal structures based on the indication;
generating a reliability metric for generating a property metric for each crystal structure in the set of crystal structures;
calculating the property metric for each crystal structure in the set of crystal structures to generate a set of energy metrics, by using a ground truth calculation of the crystal structure if the reliability metric for the crystal structure is not within a predetermined threshold;
storing the indication, the set of crystal structures, the reliability metric, the property metric, or any combination thereof in a training dataset; and
training a machine learning algorithm using the training dataset, wherein the machine learning algorithm is used to perform one or more of:
providing an indication of the one or more molecules;
generating a set of crystal structures based on the indication;
generating a reliability metric for generating a property metric for each crystal structure in the set of crystal structures; and
calculating the property metric for each crystal structure in the set of crystal structures to generate a set of energy metrics, by using a ground truth calculation of the crystal structure if the reliability metric for the crystal structure is not within the predetermined threshold.

21. One or more non-transitory computer storage media encoded with computer program instructions that when executed by one or more computers cause the one or more computers to perform a method for reporting a set of crystal structure indications for one or more molecules, comprising;
providing an indication of the one or more molecules;
generating a set of crystal structures based on the indication;
generating a reliability metric of a machine learning algorithm for generating a property metric for each crystal structure in the set of crystal structures;
calculating the property metric for each crystal structure in the set of crystal structures to generate a set of property metrics, by (i) using the machine learning algorithm if the reliability metric for the crystal structure 1s within a predetermined threshold, and (ii) using a ground truth calculation of the crystal structure if the reliability metric for the crystal structure is not within the predetermined threshold; and
reporting the set of crystal structure indications for the one or more molecules, wherein the set of crystal structure indications is based on the set of crystal structures and the set of property metrics.

22. A system comprising:
one or more computers and one or more storage devices on which are stored instructions that are operable, when executed by the one or more computers, to cause the one or more computers to perform a method for reporting a set of crystal structure indications for one or more molecules, comprising:
providing an indication of the one or more molecules;
generating a set of crystal structures based on the indication;
generating a reliability metric of a machine learning algorithm for generating a property metric for each crystal structure in the set of crystal structures;
calculating the property metric for each crystal structure in the set of crystal structures to generate a set of property metrics, by (i) using the machine learning algorithm if the reliability metric for the crystal structure 1s within a predetermined threshold, and (ii) using a ground truth calculation of the crystal structure if the reliability metric for the crystal structure is not within the predetermined threshold; and
reporting the set of crystal structure indications for the one or more molecules, wherein the set of crystal structure indications is based on the set of crystal structures and the set of property metrics.
